(19) United States
(12) Patent Application Publication
(10) Pub. No.: US 2006/0140931 A1
(43) Pub. Date: Jun. 29, 2006

(54) Bispecific molecule comprising an anti-cr1 antibody cross-linked to an antigen-binding antibody fragment

(76) Inventors: Nehal Mohamed, Budd Lake, NJ (US); Leslie S. Casey, New York, NY (US); James P. Porter, Sparta, NJ (US); Xiaoliang Wang, Pine Brook, NJ (US); Muctarr Sesay, Margate, FL (US)

Correspondence Address: LAHIVE & COCKFIELD, 28 STATE STREET, BOSTON, MA, 02109, US

(21) Appl. No.: 10/527,937

(22) PCT Filed: Sep. 16, 2003
(86) PCT No.: PCT/US03/29065

§ 371 Date: Dec. 20, 2005

Related U.S. Application Data
(60) Provisional application No. 60/411,421, filed on Sep. 16, 2002.

(51) Int. Cl. A61K 39/395; C07K 16/28

(52) U.S. Cl. 424/133.1; 530/387.3

(57) ABSTRACT

The invention provides a bispecific molecule comprising an antibody that binds a C3b-like to one or more antigen-binding antibody fragments, each of which binds an antigenic molecule. The invention also provides methods of producing such bispecific molecules and to therapeutic uses of such bispecific molecules. The invention further provides bispecific molecules in which the antigen-binding antibody fragment binds the protective antigen protein of Bacillus anthracis (Anthrax) exotoxin for treatment of Anthrax infection.

This application claims the benefit under 35 U.S.C. § 119(e) of U.S. Provisional Patent Application No. 60/411,421, filed on Sep. 16, 2002, which is incorporated by reference herein in its entirety.

### 1. FIELD OF THE INVENTION

The invention relates to a bispecific molecule comprising an antibody that binds a C3b-like receptor cross-linked to one or more antigen-binding antibody fragments, each of which binds an antigenic molecule. The invention also relates to methods of producing such bispecific molecules and to therapeutic uses of such bispecific molecules.

### 2. BACKGROUND OF THE INVENTION

Primate erythrocytes, or red blood cells (RBC's), play an essential role in the clearance of antigens from the circulatory system. The formation of an immune complex in the circulatory system activates the complement factor C3b in primates and leads to the binding of C3b to the immune complex. The C3b/immune complex then binds to the type 1 6 complement receptor (CR1), a C3b receptor, expressed on the surface of erythrocytes via the C3b molecule attached to the immune complex. The immune complex is then chaperoned by the erythrocyte to the reticuloendothelial system (RES) in the liver and spleen for neutralization. The RES cells, most notably the fixed-tissue macrophages in the liver called Kupffer cells, recognize the C3b/immune complex and break this complex from the RBC by severing the C3b receptor-RBC junction, producing a liberated erythrocyte and a C3b/immune complex which is then engulfed by the Kupffer cells and is completely destroyed within subcellular organelles of the Kupffer cells. This pathogen clearance process, however, is complement-dependent, i.e., confined to immune complexes recognized by the C3b receptor, and is ineffective in removing immune complexes which are not recognized by the C3b receptor.

Taylor et al. have discovered a complement independent method of removing pathogens from the circulatory system. Taylor et al. have shown that chemical crosslinking of a first monoclonal antibody (mAb) specific to a primate C3b receptor to a second monoclonal antibody specific to a pathogenic antigenic molecule creates a bispecific heteropolymeric antibody (HP) which offers a mechanism for binding a pathogenic antigenic molecule to a primate's C3b receptor without complement activation (U.S. Pat. Nos. 5,487,890; 5,470,570; and 5,879,679). Taylor also reported a HP which can be used to remove a pathogenic antigen specific autoantibody from the circulation. Such a HP, also referred to as an “Antigen-based Heteropolymer” (AHP), contains a CR1 specific monoclonal antibody cross-linked to an antigen (see, e.g., U.S. Pat. No. 5,879,679; Lindorfer, et al., 2001, Immunol Rev.183: 10-24; Lindorfer, et al., 2001, J Immunol Methods 248: 125-138; Ferguson, et. al., 1995, Arthritis Rheum 38: 190-200).

In addition to HP and AHP produced by cross-linking, bispecific molecules that have a first antigen recognition domain which binds a C3b-like receptor, e.g., a complement receptor 1 (CR1), and a second antigen recognition domain which binds an antigen can also be produced by methods that do not involve chemical cross-linking (see, e.g., PCT publication WO 02/46208; and PCT publication WO 01/80883). PCT publication WO 01/80833 describes bispecific antibodies produced by methods involving fusion of hybridoma cell lines, recombinant techniques, and in vitro reconstitution of heavy and light chains obtained from appropriate monoclonal antibodies. PCT publication WO 02/46208 describes bispecific molecules produced by protein trans-splicing.

Therapeutic monoclonal antibodies are mostly derived from murine hybridomas (see, e.g., Kohler and Milstein,1975, Nature 256:495-497; Cole et al., 1985, Monoclonal Antibodies and Cancer Therapy, Alan R. Liss, Inc., pp. 77-96; U.S. Pat. No. 5,914,112; and Goding, 1986, Monoclonal Antibodies: Principles and Practice, pp. 59-103, Academic Press). Such murine mAbs may trigger undesirable immune response in human patients. To circumvent the immunogenic problem associated with murine mAbs, the murine mAbs are “humanized” by, e.g., combining a variable region derived from a murine mAb with a human immunoglobulin constant region (see, e.g., van Dijk et al., Current Opinion in Chem. Biol. 5:368-374; Morrison, et al., 1984, Proc. Natl. Acad. Sci., 81, 6851-6855; Neuberger, et al., 1984, Nature 312, 604-608; Takeda, et al., 1985, Nature, 314, 452-454; U.S. Pat. Nos. 4,816,567; 4,816,397; 5,585,089; 5,225,539). Various methods have been developed to generate humanized mAbs. However, generating humanized mAbs is often a tedious process. The generated humanized mAbs may still be immunogenic (see, e.g., van Dijk et al., 2001, Current Opinion in Chem. Biol. 5:368-374).

In the last decade or so, significant progresses have been made in producing antibody fragments containing antigen-binding domains. For example, phage display libraries having a large and diverse population of specificities can be routinely generated and screened to identify high affinitive antibody fragments for a wide range of antigens (see, e.g., Watkins et al., Vox Sanguinis 78:72-79; U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734; and McCafferty et al., 1990, Nature 348:552-554). Unlike monoclonal antibodies produced by the hybridoma method, human antibody fragments can be obtained by using phage display libraries constructed from human V gene sequences. The nucleic acids encoding the antibody fragment or fragments selected from a phage display library can be obtained conveniently for construction of expression vectors. The antibody fragment or fragments can then be efficiently produced recombinantly in a variety of host systems, including bacterial and yeast (see, e.g., Plückthun et al., Immunotechnology 3:83-105; Adair, Immunological Reviews 130:5-40; Cabilly et al, U.S. Pat. No. 4,816,567; and Carter, U.S. Pat. No. 5,648,237).

Antibody fragments such as those obtained from a phage display library can have many advantageous characteristics as compared to antibodies generated by immunization of animals, such as improved affinity, wider range of specificities, and reduced immunogenic effect. However, such antibody fragments have only limited uses as therapeutics. For example, due to a lack of Fc domains, an antibody fragment cannot trigger effector functions. Antibody fragments are also cleared from the blood much more rapidly than full antibodies. Therefore, such antibody fragments are often further engineered into full antibodies. The process can be time consuming, and is not always successful.

Therefore, methods and compositions taking advantage of the binding specificities offered by an antigen-binding antibody fragment while avoiding many of their disadvantages is desirable.

Discussion or citation of a reference herein shall not be construed as an admission that such reference is prior art to the present invention.

### 3. SUMMARY OF THE INVENTION

The present invention provides bispecific molecules comprising an antibody that binds a C3b-like receptor cross-linked with an antigen-binding antibody fragment that binds an antigenic molecule. The invention also provides methods of producing the bispecific molecules of the invention and methods of therapeutic uses of the bispecific molecules of the invention.

The bispecific molecule of the invention comprises an antibody, which binds a C3b-like receptor, cross-linked via a chemical cross-linker to one or more antigen-binding antibody fragments, each of which binds an antigenic molecule. Preferably, the one or more antigen-binding antibody fragments in the bispecific molecule do not comprise an Fc domain. In a preferred embodiment, the one or more antigen-binding antibody fragments in the bispecific molecule comprise an antigen-binding antibody fragment selected from the group consisting of an Fab, an Fab′, an (Fab′)₂, and an Fv fragment of an immunoglobulin molecule. In another preferred embodiment, the one or more antigen-binding antibody fragments in the bispecific molecule comprise an single-chain Fv fragment or single-chain antibody consisting of a single chain Fv fused with a constant domain of immunoglobulin (see, e.g., Maynard et al., Nature Biotechnology 20:597-601). In still another preferred embodiment, at least one of the antigen-binding antibody fragments in the bispecific molecule is a fusion protein comprises a linker peptide fused to a Fab, Fab′, (Fab′)₂, or Fv fragment, where the linker peptide is covalently bound to the chemical cross-linker.

Preferably, the antigenic molecule that the bispecific molecule of the invention binds is a molecule desired to be removed from the circulation of a mammal. More preferably, the mammal is a human, and the antibody in the bispecific molecule binds CR1. In other preferred embodiments, the antigenic molecule that the bispecific molecule of the invention binds is an antigen of a pathogen, e.g., a bacterium or a virus. In still other preferred embodiments, the antigenic molecule that the bispecific molecule of the invention binds is a toxin.

In other preferred embodiments, at least one of the antigen-binding antibody fragments in the bispecific molecule is cross-linked at a predetermined site to the antibody that binds the C3b-like receptor. In one embodiment, the predetermined site is a cysteine residue in the antigen-binding antibody fragment. In a preferred embodiment, the predetermined site is the C-terminus of the antigen-binding antibody fragment.

In one embodiment, the antibody that binds a C3b-like receptor is a monoclonal antibody, such as a murine monoclonal antibody, e.g., murine anti-CR1 antibody 7G9, a humanized monoclonal antibody, or a human monoclonal antibody.

In a specific embodiment, the one or more antigen-binding antibody fragments bind the protective antigen (PA) protein of Bacillus anthracis (Anthrax). The one or more antigen-binding antibody fragments can be Fab fragments of murine monoclonal antibody 14B7 or single chain antibody fragments derived from murine monoclonal antibody 14B7, e.g., single chain antibody fragments consisting of a single chain Fv of 14B7 fused with a human constant k domain.

In preferred embodiments, the bispecific molecule of the invention binds its target antigenic molecule with an activity at least 5%, 15%, 25%, 50%, 90% or 99% of that of the antibody from which the antigen-binding antibody fragment is derived. In another preferred embodiments, the bispecific molecule of the invention binds its target antigenic molecule with an activity at least 5%, 15%, 25%, 50%, 90% or 99% of that of the antigen-binding antibody fragment not cross-linked with the antibody that binds a C3b-like receptor.

The invention also provides a polyclonal population of bispecific molecules comprising a plurality of different bispecific molecules, each of which comprising an antibody, which binds a C3b-like receptor, cross-linked via a chemical cross-linker to one or more antigen-binding antibody fragments, each of which binds an antigenic molecule. Preferably, the one or more antigen-binding antibody fragments in the bispecific molecule do not comprise an Fc domain. In a preferred embodiment, the one or more antigen-binding antibody fragments in the bispecific molecule comprise an antigen-binding antibody fragment selected from the group consisting of an Fab, an Fab′, an (Fab′)₂, and an Fv fragment of an immunoglobulin molecule. In another preferred embodiment, the one or more antigen-binding antibody fragments in the bispecific molecule comprise a single-chain Fv fragment or a single-chain antibody consisting of a single-chain Fv fused with a constant domain, e.g., constant k domain, of an immunoglobulin molecule. In still another preferred embodiment, at least one of the antigen-binding antibody fragments in the bispecific molecule is a fusion protein comprises a linker peptide fused to a Fab, Fab′, (Fab′)₂, or Fv fragment, where the linker peptide is covalently bound to the chemical cross-linker.

The invention also provides a method of producing a bispecific molecule, comprising cross-linking an antibody, which binds a C3b-like receptor, with an antigen-binding antibody fragment, which binds an antigenic molecule.

In a preferred embodiment, the invention provides a method of producing a bispecific molecule, which comprises (a) producing a thiol-derivatized antigen-binding antibody fragment such that said antigen-binding antibody fragment comprises a free thiol; (b) producing a maleimide-derivatized antibody that binds a C3b-like receptor such that said antibody comprises a maleimide; and (c) contacting said antigen-binding antibody fragment containing said free thiol with said antibody containing said maleimide under conditions such that said antibody and said antigen-binding antibody fragment cross-link via said maleimide and said free thiol; thereby producing said bispecific molecule. In a preferred embodiment, the antigen-binding antibody fragment is derivatized with N-succinimidyl-S-acetyl-thioacetate (SATA). In preferred embodiments, the antigen-binding antibody fragment is derivatized using SATA at a molar ratio of about 1:3 to about 1:6 antigen-binding antibody fragment:SATA. In another preferred embodiment, the antibody that binds a C3b-like receptor is derivatized with sulfosuccinimidyl 4-(N-maleimidomethyl) cyclohexane-1-carboxylate. In still another preferred embodiment, the antibody that binds a C3b-like receptor is derivatized with NHS-poly(ethylene glycol)-maleimide. In a preferred embodiment, the thiol-derivatized antigen-binding antibody fragment is mixed with the maleimide-derivatized antibody that binds a C3b-like receptor at a molar ratio of about 1:1 or 2:1 to produce the bispecific molecule of the invention.

In another preferred embodiment, the invention provides a method of producing a bispecific molecule, which comprises (a) producing an antigen-binding antibody fragment comprising a cysteine residue by a host cell such that said cysteine residue in said antigen-binding antibody fragment is maintained as a free thiol; (b) recovering said antigen-binding fragment having said free thiol; and (c) contacting said antigen-binding antibody fragment having said free thiol with a derivatized antibody that binds a C3b-like receptor under appropriate conditions such that said derivatized antibody cross-links to said antigen-binding antibody fragment at said free thiol; thereby producing said bispecific molecule. In a preferred embodiment, the antigen-binding antibody fragment containing a free thiol is secreted by the host cell. In another preferred embodiment, the derivatized antibody that binds a C3b-like receptor is derivatized with a maleimide, e.g., sulfosuccinimidyl 4-(N-maleimidomethyl) cyclohexane-1-carboxylate or NHS-poly(ethylene glycol)-maleimide. In a preferred embodiment, the antigen-binding antibody fragment containing a free thiol is mixed with the maleimide-derivatized antibody that binds a C3b-like receptor at a molar ratio of about 1:1 or 2:1 to produce the bispecific molecule of the invention.

In still another preferred embodiment, the invention provides a method of producing a bispecific molecule, comprising (a) producing a maleimide-derivatized antigen-binding antibody fragment such that said antigen-binding antibody fragment comprises a maleimide; (b) producing a thiol-derivatized antibody that binds a C3b-like receptor such that said antibody comprises a free thiol; and (c) contacting said antigen-binding antibody fragment containing said maleimide with said antibody containing said free thiol under conditions such that said antibody and said antigen-binding antibody fragment cross-link via said maleimide and said free thiol; thereby producing said bispecific molecule. In one embodiment, the antigen-binding antibody fragment is derivatized with sulfosuccinimidyl 4-(N-maleimidomethyl) cyclohexane-1-carboxylate (sSMCC). In a preferred embodiment, the antigen-binding antibody fragment is derivatized at a molar ratio of about 1:5 antigen-binding antibody fragment:sSMCC. In another embodiment, the antibody that binds a C3b-like receptor is derivatized with N-succinimidyl-S-acetyl-thioacetate (SATA). In a preferred embodiment, the antibody that binds a C3b-like receptor is derivatized with N-succinimidyl-S-acetyl-thioacetate (SATA) at a molar ratio of about 1:12 antibody:SATA. Preferably, the step (c) is carried out by a method comprising mixing said maleimide-derivatized antigen-binding antibody fragment and said thiol-derivatized antibody that binds a C3b-like receptor at a molar ratio of about 3.75:1 maleimide-derivatized antigen-binding antibody fragment:thiol-derivatized antibody.

The invention also provides the product as produced by any one of the methods of the invention.

The invention further provides a method of treating a mammal having an undesirable condition associated with the presence of an antigenic molecule in its circulation. The method comprises the step of administering to the mammal a therapeutically effective amount of a bispecific molecule comprising an antibody, which binds a C3b-like receptor, cross-linked via a chemical cross-linker to one or more antigen-binding antibody fragments, each of which binds the antigenic molecule. Any bispecific molecules of the invention can be used for this purpose. In preferred embodiments, the method is for treating a human, and the antibody in the bispecific molecule binds CR1. In other preferred embodiments, the method is for removing a pathogen, e.g., a bacterium or a virus, from the circulation of the mammal, e.g., a human by using a bispecific molecule that binds an antigen of the pathogen. In still other preferred embodiments, the method is for removing a toxin from the circulation of the mammal, e.g., a human, by using a bispecific molecule that binds the toxin.

The invention also provides a pharmaceutical composition for treating a mammal having an undesirable condition associated with the presence of an antigenic molecule in its circulation. The therapeutic composition comprises a therapeutically effective amount of a bispecific molecule of the invention and a pharmaceutically acceptable carrier.

### 4. BRIEF DESCRIPTION OF FIGURES

FIGS. 1A-1B depict exemplary processes for cross-linking 14B7Fab and 7G9 using SATA and SMCC. FIG. 1A shows a process using 1:1 conjugation. FIG. 1B shows a process using 2:1 conjugation. FIG. 1C shows a photograph of a Tris-Glycine SDS PAGE containing the 1:1 and 2:1 conjugations of a bispecific molecule 14B7Fab-SMCC-7G9 (lane 4 and 7, respectively).

FIG. 2A depicts an exemplary process for cross-linking 14B7scAb and 7G9 using SATA and NHS-PEG-MAL using 2:1 conjugation. FIG. 2B shows a photograph of a Tris-Glycine SDS PAGE containing the produced bispecific molecule 14B7scAb-PEG-7G9 (lanes 2 and 8).

FIG. 3A depicts an exemplary process for cross-linking 14B7Fab and 7G9 using SATA and NHS-PEG-MAL using 2:1 conjugation. FIG. 3B shows a photograph of a Tris-Glycine SDS PAGE containing the produced bispecific molecule 14B7Fab-PEG-7G9 (lane 7).

FIG. 4 shows survival curves plotted against the concentration of antibody samples.

### 5. DETAILED DESCRIPTION OF THE INVENTION

The present invention provides bispecific molecules comprising an antibody that binds a C3b-like receptor cross-linked with an antigen-binding antibody fragment that binds an antigenic molecule, including but not limited to a molecule comprising an epitope of a pathogen. The invention also provides methods of producing the bispecific molecules of the invention as well as methods of therapeutic uses of the bispecific molecules of the invention.

### 5.1. Bispecific Molecules

A bispecific molecule generally refers to a molecule having two or more different antigen binding specificities. The bispecific molecule of the present invention refers to a molecule comprising an anti-CR1 antibody portion that binds a C3b-like receptor, such as the type 1 complement receptor (CR1 receptor) in primates, and an antigen-binding antibody fragment portion that binds a pathogenic antigenic molecule, such as but is not limited to an epitope of a pathogen.

As used herein, the term “C3b-like receptor” refers to any mammalian circulatory molecule expressed on the surface of a mammalian blood cell, which has an analogous function to a primate C3b receptor, the CR1, in that it binds to a molecule associated with an immune complex, which is then chaperoned by the blood cell to, e.g., a phagocytic cell for clearance. As used herein, “epitope” refers to an antigenic determinant, i.e., a region of a molecule that provokes an immunological response in a host or is bound by an antibody. This region can but need not comprise consecutive amino acids. The term epitope is also known in the art as “antigenic determinant.” An epitope may comprise as few as three amino acids in a spatial conformation which is unique to the immune system of the host. Generally, an epitope consists of at least five such amino acids, and more usually consists of at least 8-10 such amino acids. Methods for determining the spatial conformation of such amino acids are known in the art. As used herein, an antigen-binding antibody fragment refers to a fragment of an antibody which is less than a full antibody and which comprises the antigen binding domain of the antibody. In the present invention, the antibody portion and the antigen-binding antibody fragment portion are linked covalently by a linker.

In the present invention, the anti-CR1 antibody portion of the bispecific molecule can be any antibody that contains a CR1 binding domain and an effector domain. In a preferred embodiment, the anti-CR1 antibody portion is an anti-CR1 monoclonal antibody (mAb). In a preferred embodiment, the anti-CR1 monoclonal antibody is 7G9, HB8592, 3D9, 57F, or 1B4 (see, e.g., Talyor et al., U.S. Pat. No. 5,487,890, which is incorporated herein by reference in its entirety). In another embodiment, the anti-CR1 antibody portion is an anti-CR1 polypeptide antibody, including but is not limited to, a single-chain variable region fragment (scFv) with specificity for a C3b-like receptor fused to the N-terminus of an immunoglobulin Fc domain. The anti-CR1 antibody portion can also be a chimeric antibody, such as but is not limited to a humanized monoclonal antibody in which the complementarity determining regions are mouse, and the framework regions are human thereby decreasing the likelihood of an immune response in human patients treated with the antibody (U.S. Pat. Nos. 4,816,567, 4,816,397, 5,693,762; 5,585,089; 5,565,332 and 5,821,337, each of which is incorporated herein by reference in its entirety). Preferably, the Fc domain of the chimeric antibody can be recognized by the Fc receptors on phagocytic cells, thereby facilitating the transfer and subsequent proteolysis of the immune complex. Although, for simplicity, this disclosure often makes references to an anti-CR1 antibody, it will be understood by a skilled artisan that the disclosure is equally applicable to antibodies that binds other C3b-like receptors.

In the present invention, the antigen-binding antibody fragment portion of the bispecific molecule can be any antigen binding fragment of an antibody which recognizes and binds an antigenic molecule. Preferably, the antigen-binding antibody fragment does not comprise an Fc domain. In a preferred embodiment, the antigen-binding antibody fragment is an Fab, an Fab′, an (Fab′)₂, or an Fv fragment of an immunoglobulin molecule. Such an Fab, Fab′ or Fv fragment can be obtained, e.g., from a full antibody by enzymatic processing or from a phage display library by affinity screening and subsequent recombinant expressing (see, e.g., Watkins et al., Vox Sanguinis 78:72-79; U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734; and McCafferty et al., 1990, Nature 348:552-554, each of which is incorporated herein by reference in its entirety). In another preferred embodiment, the antigen-binding antibody fragment is a single chain Fv (scFv) fragment which can be obtained, e.g., from a library of phage-displayed antibody fragments by affinity screening and subsequent recombinant expressing. In still another embodiment, the antigen-binding antibody fragment portion of the bispecific molecule is a single-chain antibody (scAb). As used herein, a single-chain antibody (scAb) includes antibody fragments consisting of an scFv fused with a constant domain, e.g., the constant k domain, of a immunoglobulin molecule. In another embodiment, the antigen-binding antibody fragment portion of the bispecific molecule is an Fab, Fab′, (Fab′)₂, Fv, scFv, or scAb fragment fused with a linker peptide of a desired length comprising a chosen amino acid sequence. In preferred embodiment, the linker peptide consists of 1, 2, 5, 10, or 20 amino acids. The antigenic molecule that the antigen-binding antibody fragment binds can be any substance that is present in the circulation that is potentially injurious to or undesirable in the subject to be treated, including but is not limited to proteins or drugs or toxins, autoantibodies or autoantigens, or a molecule of any infectious agent or its products. An antigenic molecule is any molecule containing an antigenic determinant (or otherwise capable of being bound by a binding domain) that is or is part of a substance (e.g., a pathogen) that is the cause of a disease or disorder or any other undesirable condition.

In preferred embodiments of the invention, the bispecific molecule comprises an anti-CR1 mAb cross-linked to one or more antigen-binding antibody fragments, such as but not limited to Fab, Fab′, (Fab′)₂, Fv, scFv, or scAb fragments. In preferred embodiments, the bispecific molecule comprises an anti-CR1 mAb cross-linked to at least 1, 2, 3, 4, 5 or 6 antigen-binding antibody fragments. Preferably, the antigen-binding antibody fragments are attached to the anti-CR1 antibody in such a way that their ability to bind the target antigen is not compromised. In preferred embodiments, the bispecific molecule of the invention binds its target antigenic molecule with an activity at least 5%, 15%, 25%, 50%, 90% or 99% of that of the antibody from which the antigen-binding antibody fragment is derived. In another preferred embodiments, the bispecific molecule of the invention binds its target antigenic molecule with an activity at least 5%, 15%, 25%, 50%, 90% or 99% of that of the antigen-binding antibody fragment not cross-linked with the antibody that binds a C3b-like receptor. In one embodiment, the antigen-binding antibody fragment is attached at a predetermined site to the anti-CR1 antibody. Preferably, such a predetermined site is selected so that the antigen-binding antibody fragment's antigen-binding affinity is not comprised. More preferably, such a predetermined site is a site on the surface of the antigen-binding fragment. In a preferred embodiment, the antigen-binding antibody fragment is attached to the anti-CR1 antibody via a cysteine residue in the antigen-binding antibody fragment. In another preferred embodiment, the cysteine via which the antigen-binding antibody fragment is attached to the anti-CR1 antibody is at the C-terminus of the antigen-binding antibody fragment.

If more than one antigen-binding antibody fragments are cross-linked to one anti-CR1 antibody, the antigen-binding antibody fragments can be the same or different. In embodiments in which the more than one antigen-binding antibody fragments are different antigen-binding antibody fragments, such antigen-binding antibody fragments can bind the same antigenic molecule. The different antigen-binding antibody fragments can also bind different antigenic molecules.

The anti-CR1 antibody, e.g., anti-CR1 mAb, and the antigen-binding antibody fragment(s) are preferably conjugated by cross-linking via a cross-linker. Any cross-linking chemistry known in art for conjugating proteins can be used in the conjunction with the present invention. In a preferred embodiment of the invention, the anti-CR1 mAb and the antigen-binding antibody fragment are produced using cross-linking agents sulfosuccinimidyl 4-(N-maleimidomethyl) cyclohexane-1-carboxylate (sSMCC) and N-succinimidyl-S-acetyl-thioacetate (SATA). In another preferred embodiment of the invention, the anti-CR1 mAb and the antigen-binding antibody fragment are conjugated via a poly-(ethylene glycol) cross-linker (PEG). In this embodiment, the PEG moiety can have any desired length. For example, the PEG moiety can have a molecular weight in the range of 200 to 20,000 Daltons. Preferably, the PEG moiety has a molecular weight in the range of 500 to 1000 Daltons or in the range of 1000 to 8000 Daltons, more preferably in the range of 3250 to 5000 Daltons, and most preferably about 5000 Daltons. Such a bispecific molecule can be produced using cross-linking agents N-succinimidyl-S-acetyl-thioacetate (SATA) and a poly(ethylene glycol)-maleimide, e.g., monomethoxy poly(ethylene glycol)-maleimide (mPEG-MAL) or NHS-poly(ethylene glycol)-maleimide (PEG-MAL). Methods of producing PEG-linked bispecific molecules is described in U.S. Provisional Application No. 60/411,731, filed on Sep. 16, 2002.

In still another preferred embodiment, the antigen-binding antibody fragment is produced with a free thiol by an appropriate host cell (see, e.g., Carter, U.S. Pat. No. 5,648,237, which is incorporated herein by reference in its entirety), and the bispecific molecule is produced by reacting the free thiol containing antibody fragment with an appropriately derivatized, e.g., sSMCC derivatized, anti-CR1 mAb. An anti-CR1 antibody with a free thiol can also be produced directly, i.e., without using a chemical cross-linker, e.g., a maleimide. Thus, in another preferred embodiment, the bispecific molecule comprises a monoclonal anti-CR1 antibody conjugated with an antigen-binding antibody fragment via a disulfide bond. Such a bispecific molecule can be produced by mixing an antigen-binding antibody fragment having a free thiol with an anti-CR1 antibody with a free thiol.

The invention also provides a polyclonal population of bispecific molecules, each comprising an antibody that binds a C3b-like receptor cross-linked with a different antigen-binding antibody fragment that binds an antigenic molecule. A polyclonal population of bispecific molecules of the present invention refers broadly to any population comprising a plurality of different bispecific molecules, each of which comprising an antibody that binds a C3b-like receptor cross-linked to a different antigen-binding antibody fragment that binds a pathogenic antigenic molecule. The population thus comprises a plurality of different bispecific molecules having a plurality of different antigen binding specificities via the different antibody fragments. The plurality of different antibody fragments can recognize and bind the same epitope on a pathogen. The plurality of different antigen binding specificities can also be directed to a plurality of different epitopes on a pathogen. The plurality of different antigen binding specificities can also be directed to a plurality of variants of a pathogen. The plurality of different antigen binding specificities can further be directed to a plurality of different pathogens. The plurality of different antigen recognition of specificities can further be directed to a plurality of different epitopes on a plurality of different pathogens. The characteristic and function of each member bispecific molecule in the plurality of bispecific molecules in the polyclonal population can be known or unknown. The exact proportion of each member bispecific molecule in the plurality of bispecific molecules in the polyclonal population can also be known or unknown. Preferably, the characteristics and the proportions of at least some member bispecific molecules in the plurality of bispecific molecules in the polyclonal population are known so that if desired, the exact proportions of such members can be adjusted for optimal therapeutic and/or prophylactic efficacy. The polyclonal population of bispecific molecules can comprise bispecific molecules that do not bind the target pathogenic antigenic molecule or pathogenic antigenic molecules. For example, the population of bispecific molecules can be prepared from a hyperimmune serum that contains antibodies that bind antigenic molecules other than those that are on the target pathogens. Preferably, the plurality of bispecific molecules in the polyclonal population constitutes at least 1%, 5%, 10%, 20%, 50% or 80% of the population. More preferably, the plurality of bispecific molecules in the polyclonal population constitutes at least 90% of the population. The plurality of bispecific molecules in the polyclonal population of bispecific molecules preferably does not comprise any single bispecific molecule which has a proportion exceeding 95%, 80%, or 60% of the plurality. More preferably, the plurality of bispecific molecules in the polyclonal population of bispecific molecules does not comprise any single bispecific molecule which has a proportion exceeding 50% of the plurality. The plurality of bispecific molecules in the polyclonal population comprises at least 2 different bispecific molecules with different antigen binding specificities. Preferably, the plurality of bispecific molecules in the polyclonal population comprises at least 10 different bispecific molecules with different antigen binding specificities. More preferably, the plurality of bispecific molecules in the polyclonal population comprises at least 100 different bispecific molecules with different antigen binding specificities. The polyclonal population can be a polyclonal population generated from a suitable polyclonal population of antigen recognition portions, such as but is not limited to a polyclonal immunoglobulin preparation.

### 5.2.1. Production of Anti-CR1 Antibodies

The term “antibody” as used herein refers to immunoglobulin molecules. The immunoglobulin molecules are encoded by genes which include the kappa, lambda, alpha, gamma, delta, epsilon and mu constant regions, as well as a myriad of immunoglobulin variable regions. Light chains are classified as either kappa or lambda. Light chains comprise a variable light (V_{L}) and a constant light (C_{L}) domain. Heavy chains are classified as gamma, mu, alpha, delta, or epsilon, which in turn define the immunoglobulin classes IgG, IgM, IgA, IgD and IgE, respectively. Heavy chains comprise variable heavy (V_{H}), constant heavy 1 (CH1), hinge, constant heavy 2 (CH2), and constant heavy 3 (CH3) domains. The IgG heavy chains are further sub-classified based on their sequence variation, and the subclasses are designated IgG1, IgG2, IgG3 and IgG4.

Antibodies can be further broken down into two pairs of a light and heavy domain. The paired V_{L} and V_{H} domains each comprise a series of seven subdomains: framework region 1 (FR1), complementarity determining region 1 (CDR1), framework region 2 (FR2), complementarity determining region 2 (CDR2), framework region 3 (FR3), complementarity determining region 3 (CDR3), framework region 4 (FR4) which constitute the antibody-antigen recognition domain.

A chimeric antibody may be made by splicing the genes from a monoclonal antibody of appropriate antigen specificity together with genes from a second human antibody of appropriate biologic activity. More particularly, the chimeric antibody may be made by splicing the genes encoding the variable regions of an antibody together with the constant region genes from a second antibody molecule. This method is used in generating a humanized monoclonal antibody wherein the complementarity determining regions are mouse, and the framework regions are human thereby decreasing the likelihood of an immune response in human patients treated with the antibody (U.S. Pat. Nos. 4,816,567, 4,816,397, 5,693,762; 5,585,089; 5,565,332 and 5,821,337, each of which is incorporated herein by reference in its entirety).

An antibody suitable for use in the present invention may be obtained from natural sources or produced by hybridoma, recombinant or chemical synthetic methods, including modification of constant region functions by genetic engineering techniques (United States Pat. No. 5,624,821). The antibody of the present invention may be of any isotype, but is preferably human IgG1.

An antibody can also be a single-chain antibody (scFv) which generally comprises a fusion polypeptide consisting of a variable domain of a light chain fused via a polypeptide linker to the variable domain of a heavy chain.

An anti-CR1 mAb that binds a human C3b receptor can be produced by known methods. In one embodiment, anti-CR1 mAb, preferably an anti-CR1 IgG, can be prepared using standard hybridoma precedure known in the art (see, for example, Kohler and Milstein, 1975, Nature 256:495-497; Hogg et al., 1984, Eur. J. Immunol. 14:236-243; O'Shea et al., 1985, J. Immunol. 134:2580-2587; Schreiber, U.S. Pat. No. 4,672,044). A suitable mouse is immunized with human CR1 which can be purified from human erythrocytes. The spleen cells obtained from the immunized mouse are fused with an immortal mouse myeloma cell line which results in a population of hybridoma cells, including a hybridoma that produces an anti-CR1 antibody. The hybridoma which produces the anti-CR1 antibody is then selected, or ‘cloned’, from the population of hybridomas using conventional techniques such as enzyme linked immunosorbent assays (ELISA). Hybridoma cell lines expressing anti-CR1 mAb can also be obtained from various sources, for example, the murine anti-CR1 mAb that binds human CR1 described in U.S. Pat. No. 4,672,044 is available as hybridoma cell line ATCC HB 8592 from the American Type Culture Collection (ATCC). The obtained hybridoma cells are grown and washed using standard methods known in the art. Anti-CR1 antibodies are then recovered from supernatants.

In other embodiments, nucleic acids encoding the heavy and light chains of an anti-CR1 mAb, preferably an anti-CR1 IgG, are prepared from the hybridoma cell line by standard methods known in the art. As a non-limiting example, cDNAs encoding the heavy and light chains of the anti-CR1 IgG are prepared by priming MRNA using appropriate primers, followed by PCR amplification using appropriate forward and reverse primers. Any commercially available kits for cDNA synthesis can be used. The nucleic acids are used in the construction of expression vector(s). The expression vector(s) are transfected into a suitable host. Non-limiting examples include E. coli, yeast, insect cell, and mammalian systems, such as a Chinese hamster ovary cell line. Antibody production can be induced by standard method known in the art.

An anti-CR1 antibody can be prepared by immunizing a suitable subject with human CR1 which can be purified from human erythrocytes. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique by Kozbor et al. (1983, Immunol. Today 4:72), the EBV-hybridoma technique by Cole et al. (1985, Monoclonal Antibodies and Cancer Therapy, Alan R. Liss, Inc., pp. 77-96) or trioma techniques. The technology for producing hybridomas is well known (see Current Protocols in Immunology, 1994, John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., 1975, Nature, 256:495, or may be made by recombinant DNA methods (U.S. Pat. No. 4,816,567). The term “monoclonal antibody” as used herein also indicates that the antibody is an immunoglobulin.

In the hybridoma method of generating monoclonal antibodies, a mouse or other appropriate host animal, such as a hamster, is immunized as hereinabove described to elicit lymphocytes that produce or are capable of producing antibodies that will specifically bind to the protein used for immunization (see, e.g., U.S. Pat. No. 5,914,112, which is incorporated herein by reference in its entirety.)

Alternatively, lymphocytes may be immunized in vitro. Lymphocytes are then fused with myeloma cells using a suitable fusing agent, such as polyethylene glycol, to form a hybridoma cell (Goding, Monoclonal Antibodies: Principles and Practice, pp. 59-103, Academic Press, 1986). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Human myeloma and mouse-human heteromyeloma cell lines also have been described for the production of human monoclonal antibodies (Kozbor, 1984, J. Immunol., 133:3001; Brodeur et al., Monoclonal Antibody Production Techniques and Applications, pp. 51-63 (Marcel Dekker, Inc., New York, 1987)). Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immuno-absorbent assay (ELISA). The binding affinity of the monoclonal antibody can, for example, be determined by the Scatchard analysis of Munson et al., 1980, Anal. Biochem., 107:220.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Alternative to preparing monoclonal antibody-secreting hybridomas, a monoclonal antibody directed against human CR1 can be identified and isolated by screening a recombinant combinatorial immunoglobulin library (e.g., an antibody phage display library) with human CR1. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

In addition, techniques developed for the production of “chimeric antibodies” (Morrison, et al., 1984, Proc. Natl. Acad. Sci., 81, 6851-6855; Neuberger, et al., 1984, Nature 312, 604-608; Takeda, et al., 1985, Nature, 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, each of which is incorporated herein by reference in its entirety)

Humanized antibodies are antibody molecules from non-human species having one or more complementarity determining regions (CDRs) from the non-human species and a framework region from a human immunoglobulin molecule. (see e.g., U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567 and 5,225,539; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Natl. Cancer Inst. 80:1553-1559; Morrison 1985, Science 229:1202-1207; Oi et al., 1986, Bio/Techniques 4:214; Jones et al., 1986, Nature 321:552-525; Verhoeyan et al., 1988, Science 239:1534; and Beidler et al., 1988, J. Immunol. 141:4053-4060.

Complementarity determining region (CDR) grafting is another method of humanizing antibodies. It involves reshaping murine antibodies in order to transfer full antigen specificity and binding affinity to a human framework (Winter et al. U.S. Pat. No. 5,225,539). CDR-grafted antibodies have been successfully constructed against various antigens, for example, antibodies against IL-2 receptor as described in Queen et al., 1989 (Proc. Natl. Acad. Sci. USA 86:10029); antibodies against cell surface receptors-CAMPATH as described in Riechmann et al. (1988, Nature, 332:323; antibodies against hepatitis B in Cole et al. (1991, Proc. Natl. Acad. Sci. USA 88:2869); as well as against viral antigens-respiratory syncitial virus in Tempest et al. (1991, Bio-Technology 9:267). CDR-grafted antibodies are generated in which the CDRs of the murine monoclonal antibody are grafted into a human antibody. Following grafting, most antibodies benefit from additional amino acid changes in the framework region to maintain affinity, presumably because framework residues are necessary to maintain CDR conformation, and some framework residues have been demonstrated to be part of the antigen binding site. However, in order to preserve the framework region so as not to introduce any antigenic site, the sequence is compared with established germline sequences followed by computer modeling.

Completely human antibodies are particularly desirable for therapeutic treatment of human patients. Such antibodies can be produced using transgenic mice which are incapable of expressing endogenous immunoglobulin heavy and light chain genes, but which can express human heavy and light chain genes. The transgenic mice are immunized in the normal fashion with human CR1.

Monoclonal antibodies directed against human CR1 can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.; see, for example, U.S. Pat. No. 5,985,615) and Medarex, Inc. (Princeton, N.J.), can be engaged to provide human antibodies directed against human CR1 using technology similar to that described above.

Completely human antibodies which recognize and bind a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903).

A pre-existing anti-CR1 antibody, including but not limited to 7G9, HB8592, 3D9, 57F, and IB4 (see, e.g., Talyor et al., U.S. Pat. No. 5,487,890, which is incorporated herein by reference in its entirety), can also be used. In a preferred embodiment, a hybridoma cell line secreting a high-affinity anti-CR1 monoclonal antibody, e.g., 7G9 (murine IgG₂a, kappa), is used to generate a master cell bank (MCB). Preferably, the master cell bank is tested for mouse antibody production, mycoplasma and sterility. The anti-CR1 antibody is then produced and purified from ascites fluid. In another preferred embodiment, the anti-CR1 monoclonal antibody used for the production of the bispecific molecules is produced in vitro (hollow-fiber bioreactor) and purified under cGMP.

### 5.2.2. Production of Antigen-Binding Antibody Fragments

The antigen-binding antibody fragment of the bispecific molecule of the invention can be produced by various methods known in the art.

In one embodiment, the antibody fragment is a fragment of an immunoglobulin molecule containing a binding domain which specifically binds an antigenic molecule. Examples of immunologically active fragments of immunoglobulin molecules include but are not limited to Fab, Fab′ and (Fab′)₂ fragments which can be generated by treating an appropriate antibody with an enzyme such as pepsin or papain. In a preferred embodiment, an antigen-binding antibody fragment is produced from a monoclonal antibody having the desired antigen binding specificity. Such a monoclonal antibody can be raised using the targeted antigenic molecule by any of the standard methods known in the art. For example, a monoclonal antibody directed against an antigenic molecule can be raised using any one of the methods described in Section 5.2.1., supra, using the antigenic molecule in the place of CR1. The antibody can then be treated with pepsin or papain. For example, pepsin digests an antibody below the disulfide linkages in the hinge region to produce an (Fab′)₂ fragment of the antibody which is a dimer of the Fab composed of a light chain joined to a VH-CH1 by a disulfide bond. The (Fab′)₂ fragments may be reduced under mild conditions to break the disulfide linkage in the hinge region thereby converting the (Fab′)₂ dimer to a Fab′ monomer. The Fab′ monomer is essentially an Fab with part of the hinge region. See Paul, ed., 1993, Fundamental Immunology, Third Edition (New York: Raven Press), for a detailed description of epitopes, antibodies and antibody fragments. A skilled person in the art will recognize that such Fab′ fragments may be synthesized de novo either chemically or using recombinant DNA technology. Thus, as used herein, the term antibody fragments includes antibody fragments produced by the modification of whole antibodies or those synthesized de novo.

In another embodiment, the method of generating and expressing immunologically active fragments of antibodies described in U.S. Pat. No. 5,648,237, which is incorporated herein by reference in its entirety, is used.

In still another embodiment, the antigen-binding antibody fragment, e.g., an Fv, Fab, Fab′, or (Fab′)₂ is produced by a method comprising affinity screening of a phage display library (see, e.g., Watkins et al., Vox Sanguinis 78:72-79; U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734; and McCafferty et al., 1990, Nature 348:552-554, each of which is incorporated herein by reference in its entirety). The nucleic acids encoding the antibody fragment or fragments selected from the phage display library is then obtained for construction of expression vectors. The antibody fragment or fragments can then be produced in a suitable host system, such as a bacterial, yeast, or mammalian host system (see, e.g., Plückthun et al., Immunotechnology 3:83-105; Adair, Immunological Reviews 130:5-40; Cabilly et al, U.S. Pat. No. 4,816,567; and Carter, U.S. Pat. No. 5,648,237, each of which is incorporated herein by reference in its entirety).

In still another embodiment, techniques described for the production of single chain antibodies (U.S. Pat. No. 4,946,778; Bird, 1988, Science 242:423-426; Huston et al., 1988, Proc. Natl. Acad. Sci. USA 85:5879-5883; Ward et al., 1989, Nature 334:544-546; and Maynard et al., Nature Biotechnology 20:597-601, each of which is incorporated herein by reference in its entirety) can be adapted to produce single chain antibodies against the antigenic molecule. Single chain antibodies are formed by linking the heavy and light chain fragments of the Fv region via an amino acid bridge, resulting in a single chain polypeptide. Single chain antibodies can also contain, in addition to the Fv region, a constant domain of immunoglobulin.

In a preferred embodiment, the antigen-binding antibody fragment can be modified such that it can be attached at a predetermined site to an anti-CR1 antibody. Preferably, such a predetermined site is selected so that the antigen-binding affinity is not compromised after the fragment is cross-linked to the anti-CR1 antibody. More preferably, such a predetermined site is a site on the surface of the antigen-binding antibody fragment. In a preferred embodiment, a cysteine residue is engineered into an appropriate location in an antigen-binding antibody fragment to allow site-specific attachment of the antigen-binding antibody fragment to an anti-CR1 antibody (see, e.g., Lyons et al., Protein Engineering 3:703-708, which is incorporated herein in its entirety). A skilled person in the art will be able to determine the location where the cysteine residue is introduced as well as the method that can be used to generate such an engineered fragment. In a preferred embodiment, the cysteine is introduced to the C-terminus of the antigen-binding antibody fragment.

In another preferred embodiment, the antigen-binding antibody fragment containing a cysteine residue is produced by a host cell in such a manner that a cysteinyl free thiol is maintained (see, e.g., Carter, U.S. Pat. No. 5,648,237, which is incorporated herein in its entirety). The antigen-binding antibody fragment containing cysteinyl free thiol (also referred to as “Ab-fragment-cys-SH”) can then be used to produce the bispecific molecule of the invention directly with an appropriate anti-CR1 antibody or an appropriately derivatized anti-CR1 antibody which can react with the free thiol to form a covalent bond. Anti-CR1 antibody can be a maleimide derivatized anti-CR1 monoclonal antibody, e.g., an anti-CR1 monoclonal antibody derivatized with sulfosuccinimidyl 4-(N-maleimidomethyl) cyclohexane-1-carboxylate (sSMCC) or a poly(ethylene glycol)-maleimide, e.g., monomethoxy poly(ethylene glycol)-maleimide (mPEG-MAL) or NHS-poly(ethylene glycol)-maleimide (PEG-MAL). Alternatively, the anti-CR1 antibody can be a thiolated anti-CR1 antibody, e.g., an anti-CR1 antibody derivatized with N-succinimidyl-S-acetyl-thioacetate (SATA), N-succinimidyl-3-(2-pyridyldithio)propionate (SPDP). The Ab-fragment-cys-SH can be cross-linked with the thiolated anti-CR1 antibody via a disulfide bond.

The invention also uses a polyclonal population of antigen-binding antibody fragments for production of a polyclonal population of bisepcific molecules. Any method known in the art for producing a polyclonal population of antigen-binding antibody fragments can be used in conjunction with the present invention. In preferred embodiments, a population of antigen-binding antibody fragments can be produced from a population of antibodies, e.g., a polyclonal population of antibodies, having the desired binding specificities (see, e.g., PCT publication WO 02/075275; PCT publication WO 02/46208; and PCT publication WO 01/80883, each of which is incorporated herein by reference in its entirety, for methods of producing a polyclonal population of antigen-binding antibodies). In one embodiment, a polyclonal population of antibodies can be produced by immunization of a suitable animal, such as but is not limited to mouse, rabbit, and horse.

In one embodiment, an immunogenic preparation, typically comprising the antigenic molecules, e.g., associated with the pathogen or pathogens to be cleared from a subject, are used to prepare antibodies by immunizing a suitable subject (e.g., rabbit, goat, mouse or other mammal). An appropriate immunogenic preparation can contain, for example, antigens isolated from cells or tissue sources, antigens recombinantly expressed or antigens chemically synthesized by, e.g., using standard peptide synthesis techniques. An immunogenic preparation can also contain chimeric or fusion antigens, which comprise all or part of an antigen for use in the invention, operably linked to a heterologous polypeptide, including but is not limited to a GST fusion antigen in which the antigen is fused to the C-terminus of GST sequences or an immunoglobulin fusion protein in which all or part of an antigen is fused to sequences derived from a member of the immunoglobulin protein family. Chimeric and fusion proteins can be produced by standard recombinant DNA techniques. The preparation can further include an adjuvant, such as Freund's complete or incomplete adjuvant, or similar immunostimulatory agent. A mixture of toxic substances, such as those contained in a reptile or snake bite, can also be used to raise antibody directed to such substances.

The immunogen is then used to immunize a suitable animal. Preferably, the animal is a specialized transgenic animal that can secret human antibody. Non-limiting examples include transgenic mouse strains which can be used to produce a polyclonal population of antibodies directed to a specific pathogen (Fishwild et al., 1996, Nature Biotechnology 14:845-851; Mendez et al., 1997, Nature Genetics 15:146-156). In one embodiment of the invention, transgenic mice that harbor the unrearranged human immunoglobulin genes are immunized with the target immunogens. After a vigorous immune response against the immunogen has been elicited in the mice, the blood of the mice are collected and a purified preparation of human IgG molecules can be produced from the plasma or serum. Any methods known in the art can be used to obtain the purified preparation of human IgG molecules, including but is not limited to affinity column chromatography using anti-human IgG antibodies bound to a suitable column matrix. Anti-human IgG antibodies can be obtained from any sources known in the art, e.g., from commercial sources such as Dako Corporation and ICN. The preparation of IgG molecules produced comprises a polyclonal population of IgG molecules that bind to the immunogen or immunogens at different degree of affinity. Preferably, a substantial fraction of the preparation are IgG molecules specific to the immunogen or immunogens. Although polyclonal preparations of IgG molecules are described, it is understood that polyclonal preparations comprising any one type or any combination of different types of immunoglobulin molecules are also envisioned and are intended to be within the scope of the present invention.

A polyclonal preparation of antibodies or hyperimmune serum directed to a specific pathogen or pathogens and/or pathogenic antigenic molecule or pathogenic antigenic molecules can be produced from human patients who have been infected by the pathogen or pathogens and/or the pathogenic antigenic molecule or pathogenic antigenic molecules using any methods known in the art (see, e.g., Harlow et al., Using Antibodies A Laboratory Manual). As non-limiting examples, hyperimmune serum against parasites, bacteria, and viruses can be prepared according to methods described in, e.g., Shi et al., 1999, American J Tropical Med. Hyg. 60:135-141, Cryz et al., 1986, J. Lab. Clin. Med. 108:182-189, and Cummins et al., 1991, Blood 77:1111-1117. In a preferred embodiment, a polyclonal human IgG preparation is produced using a chromatographic method as described in Tanaka et al., 1998, Brazilian Journal of Medical and Biological Research 31:1375-81, which is incorporated herein by reference in its entirety. Specifically, a combination of ion-exchange, DEAE-Sepharose FF and arginine Sepharose 4B affinity chromatography, and Sephacryl S-300 HR gel filtration is used to produce purified IgG molecules from the gamma-globulin fraction of the human plasma.

However, the present invention is not limited to polyclonal preparations of IgG molecules. It is understood that polyclonal preparations comprising any one type or any combination of different types of immunoglobulin molecules, including but are not limited 30 to IgG, IgE, IgA, etc., are also envisioned and are intended to be within the scope of the present invention. Such polyclonal preparations can be produced using any standard method known in the art. The purified polyclonal preparation is then used in the production of the polyconal population of antigen-binding antibody fragments.

A population of antigen-binding antibody fragments directed to a specific pathogenic antigenic molecule or pathogenic antigenic molecules can be produced from a phage display library. Polyclonal antigen-binding antibody fragments can be obtained by affinity screening of a phage display library having a sufficiently large and diverse population of specificities with an antigen or antigens of interest. Examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734; and McCafferty et al., 1990, Nature 348:552-554.

In a preferred embodiment, the polyclonal population of antigen-binding antibody fragments directed to a pathogenic antigenic molecule or pathogenic antigenic molecules is produced from a phage display library according to Den et al., 1999, J. Immunol. Meth. 222:45-57; Sharon et al. Comb. Chem. High Throughput Screen. 2000 3:185-96; and Baecher-Allan et al., Comb. Chem. High Throughput Screen. 2000 2:319-325. The phage display library is screened to select a polyclonal sublibrary having binding specificities directed to the antigenic molecule or antigenic molecules of interests by affinity chromatography (McCafferty et al., 1990, Nature 248:552; Breitling et al., 1991, Gene 104:147; and Hawkins et al., 1992, J. Mol. Biol. 226:889). The nucleic acids encoding the heavy and light chain variable regions are then linked head to head to generate a library of bidirectional phage display vectors. The bidirectional phage display vectors are then transferred in mass to bidirectional mammalian expression vectors (Sarantopoulos et al., 1994, J. Immunol. 152:5344) which are used to transfect a suitable hybridoma cell line. The transfected hybridoma cells are induced to produce the antigen-binding antibody fragments using any method known in the art.

In other preferred embodiments, the population of antigen-binding antibody fragments directed to a pathogenic antigenic molecule or pathogenic antigenic molecules is produced by a method using the whole collection of selected displayed antibody fragments without clonal isolation of individual members as described in U.S. Pat. No. 6,057,098, which is incorporated by reference herein in its entirety. Polyclonal antigen-binding antibody fragments are obtained by affinity screening of a phage display library having a sufficiently large repertoire of specificities with, e.g., an antigenic molecule having multiple epitopes, preferably after enrichment. of displayed library members that display multiple antibodies. The nucleic acids encoding the selected display antibody fragments are excised and amplified using suitable PCR primers. The nucleic acids can be purified by gel electrophoresis such that the full length nucleic acids are isolated. Each of the nucleic acids is then inserted into a suitable expression vector such that a population of expression vectors having different inserts is obtained. The population of expression vectors is then expressed in a suitable host.

### 5.2.3. Production of Bispecific Molecules

The bispecific molecule of the present invention can be a covalent conjugate of one or more antigen-binding antibody fragments with an anti-CR1 monoclonal antibody, e.g., the 7G9 antibody as described in U.S. Pat. No. 5,879,679. Any standard chemical cross-linking methods can be used in the present invention. Preferably, a cross-linking method employing a bifunctional cross-linker is used. Preferably, a cross-linking method employing a bifunctional poly(ethylene glycol) cross-linker is used. For example, cross-linking agents, including but not limited to, protein A, glutaraldehyde, carbodiimide, N-succinimidyl-S-acetyl-thioacetate (SATA), N-succinimidyl-3-(2-pyridyldithio)propionate (SPDP), sulfosuccinimidyl 4-(N-maleimidomethyl) cyclohexane-1-carboxylate (sSMCC), and a poly(ethylene glycol)-maleimide, e.g., monomethoxy poly(ethylene glycol)-maleimide (mPEG-MAL), NHS-poly(ethylene glycol)-maleimide (PEG-MAL), succinimidyl 6-hydrazinonicotinate acetone hydrazone (SANH) or succinimidyl 4-formyl benzoate (SFB) can be used.

In a preferred embodiment, SATA is used to derivatize an antigen-binding antibody fragment. A skilled person in the art will be able to determine the concentrations of the antigen-binding antibody fragment and SATA. In one embodiment, by way of example but not limitation, the following protocol is used. A solution of SATA in DMSO is prepared. The antigen-binding antibody fragment is dialyzed against PBSE buffer. The coupling reaction is initiated by combining the antigen-binding antibody fragment and SATA at a molar ratio of about 1:6. The reactants are mixed by inversion and incubated at room temperature for a desired period of time with mixing. A hydroxylamine HCl solution is prepared by adding hydroxyamine and EDTA to MES. The Hydroxylamine HCl solution is added to the reaction mixture from the SATA coupling step at an appropriate molar ratio, e.g., a molar ratio of about 2000:1, and incubated for a desired period of time at room temperature under argon atmosphere. The reaction mixture is then desalted by chromatography using an Amersham Hi-Prep desalting column in MES buffer. The SATA derivatized antigen-binding antibody fragment can then be used with an appropriately derivatized anti-CR1 antibody, e.g., a maleimide derivatized anti-CR1 antibody, to produce the bispecific molecule of the invention.

In another preferred embodiment, the antigen-binding antibody fragment containing a cysteine residue is produced by a host cell in such a manner that a free thiol is maintained (see, e.g., Carter, U.S. Pat. No. 5,648,237, which is incorporated herein in its entirety). Preferably, the antigen-binding antibody fragment containing a free thiol is secreted by the host cell. The antigen-binding antibody fragment containing the free thiol can then be recovered and used with an appropriately derivatized anti-CR1 antibody, e.g., a maleimide derivatized anti-CR1 antibody, to produce the bispecific molecule of the invention.

In one embodiment, the anti-CR1 antibody is derivatized with a maleimide using any method known in the art. A skilled person in the art will be able to determine the concentrations of the anti-CR1 antibody and maleimide to achieve a desired number of cross-linking sites on the anti-CR1 antibody. In a preferred embodiment, the antibody is derivatized with maleimide as follows: a fresh stock solution of sSMCC Conjugation solution is prepared in PBSE buffer; the antibody is dialyzed exhaustively against PBSE buffer; the coupling reaction is initiated by combining the antibody and sSMCC at a molar ratio of about 1:6; the reactants are mixed by inversion and incubated at room temperature for 60 min with mixing; and the sSMCC-antibody is recovered by size exclusion chromatography using FPLC with two Pharmacia 26/10 Desalting Columns in series (cat#17-5087-01). The column is preferably pre-washed with distilled water followed by PBSE buffer according to the manufacturer's instructions before loaded with the reaction mixture. The maleimide modified antibody is eluted in the void volume with PBSE buffer and should be used within 15 min. The maleimide derivatized anti-CR1 antibody can then be used with an appropriately antigen-binding antibody fragment, e.g., a SATA derivatized anti-CR1 antibody, to produce the bispecific molecule of the invention.

In another embodiment, the anti-CR1 antibody is derivatized with an poly(ethylene glycol)-maleimide, e.g., NHS-poly(ethylene glycol)-maleimide (PEG-MAL), using any method known in the art. A skilled person in the art will be able to determine the concentrations of the antibody and the PEG-MAL. In this embodiment, the PEG moiety can have any desired length. For example, the PEG moiety can have a molecular weight in the range of 200 to 20,000 Daltons. Preferably, the PEG moiety has a molecular weight in the range of 500 to 1000 Daltons or from 1000 to 8000 Daltons, more preferably in the range of 3250 to 5000 Daltons, and most preferably about 5000 Daltons. Methods of producing PEG-linked bispecific molecules is described in U.S. Provisional Application No. 60/411,731, filed on Sep. 16, 2002. In one embodiment, by way of example but not limitation, the following protocol is used. A MES solution of NHS-PEG-MAL is prepared. The NHS-PEG-MAL solution is added to anti-CR1 antibody, e.g., 7G9, at a molar ratio of about 6:1 (PEG:antibody). The reactants are mixed by inversion and incubated at room temperature for an appropriate period of time with mixing. The reaction mixture is then desalted by chromatography using an Amersham Hi-Prep desalting column in MES buffer. The PEG-maleimide derivatized anti-CR1 antibody can then be used with an appropriately antigen-binding antibody fragment, e.g., a SATA derivatized anti-CR1 antibody, to produce the bispecific molecule of the invention.

In another embodiment, the anti-CR1 antibody is thiolated, e.g., derivatized with N-succinimidyl-S-acetyl-thioacetate (SATA), N-succinimidyl-3-(2-pyridyldithio)propionate (SPDP). The thiolated anti-CR1 antibody can then be used with an appropriately antigen-binding antibody fragment, e.g., a SATA derivatized anti-CR1 antibody, to produce the bispecific molecule of the invention.

The derivatized antibody, e.g., antibody-maleimide, antibody-PEG-maleimide, or antibody-SH, and the antigen-binding antibody fragment containing a free thiol, also referred to as Ab-fragment-SH, are then combined at a desired molar ratio of derivatized-antibody:antibody-fragment. A skilled person in the art will be able to determine the molar ratio of the derivatized anti-CR1 antibody and antibody-fragment to achieve a desired number of antigen-binding antibody fragments to each anti-CR1 antibody. In a preferred embodiment, the maleimide-antibody and Ab-fragment-SH are combined at a molar ratio of about 2:1 (derivatized-antibody:Ab-fragment-SH). In another preferred embodiment, the derivatized-antibody and antibody-fragment-SH are combined at a molar ratio of about 1:1 (derivatized-antibody:Ab-fragment-SH). In preferred embodiments, 1, 2, 3, 4, 5 or 6 antigen-binding antibody fragments are conjugated to each anti-CR1 antibody.

In addition, embodiments in which the antigen-binding antibody fragment is derivatized with a maleimide, e.g., sSMCC or NHS-PEG-MAL, whereas the anti-CR1 antibody is, e.g., using SATA or SDPD, are also envisioned. In a preferred embodiment, the antigen-binding antibody fragment is derivatized with sSMCC at a molar ratio of about 1:5, and the anti-CR1 antibody is derivatized with SATA at a molar ratio of about 1:12. The obtained antibody-fragment-SMCC and antibody-SH are combined at a molar ratio of 3.75:1. In a preferred embodiment, the antigen-binding fragment is 14B7scAb and the anti-CR1 antibody is the murine monoclonal anti-CR1 antibody 7G9. The bispecific molecule 14B7scAb-7G9 produced has a molecular weight of about 140 kDalton, which corresponds to two scAb molecules cross-linked to each 7G9.

In a specific embodiment, the method of the invention is used for producing a bispecific molecule comprising an antibody that binds a C3b-like receptor cross-linked with an antigen-binding antibody fragment which binds the protective antigen (PA) protein of Bacillus anthracis (Anthrax) (see, e.g., Little et al., 1991, Biochem Biophys Res Commun.180:531-7; Little et al., 1988, Infect Immun. 56:1807-13). In one embodiment, the antibody fragment is the Fab fragment of an antibody 14B7 which binds PA. In another embodiment, the antibody fragment is a single chain antibody fragment derived from 14B7, e.g., a single-chain antibody consisting of a single chain Fv of 14B7 fused with a human constant k domain (14B7scAb). In a preferred embodiment, the antibody that binds a C3b-like receptor is the murine anti-CR1 IgG 7G9. In a preferred embodiment, the bispecific molecule is produced by cross-linking an anti-CR1 mAb, e.g., 7G9, and an anti-PA Fab fragment, e.g., 14B7Fab, using N-succinimidyl-S-acetyl-thioacetate (SATA) and sulfosuccinimidyl 4-(N-maleimidomethyl) cyclohexane-1-carboxylate (sSMCC) as the cross-linking agents. In another preferred embodiment, the bispecific molecule is produced by cross-linking an anti-CR1 mAb, e.g., 7G9, and an anti-PA single-chain antibody, e.g., 14B7scAb, using N-succinimidyl-S-acetyl-thioacetate (SATA) and NHS-poly(ethylene glycol)-maleimide (PEG-MAL) as the cross-linking agents. In still another preferred embodiment, the bispecific molecule is produced by cross-linking an anti-CR1 mAb, e.g., 7G9, and an anti-PA single chain antibody, e.g., 14B7Fab, using N-succinimidyl-S-acetyl-thioacetate (SATA) and NHS-poly(ethylene glycol)-maleimide (PEG-MAL) as the cross-linking agents. In another embodiment, a polyclonal population of bispecific molecules of the invention is produced by cross-linking an anti-CR1 antibody described in Section 5.2.1, supra, and a polyclonal population of antigen-binding antibody fragments described in Section 5.2.2, supra, by a method described in this section.

### 5.2.4. Purification and Characterization of Bispecific Molecules

The bispecific molecules produced by a method such as described supra are then preferably purified. Bispecific molecules can be purified by any method known to one skilled in the art using molecular size or specific binding affinity or a combination thereof. In one embodiment, the bispecific molecules can be purified by ion exchange chromatography using columns suitable for isolation of the bispecific molecules of the invention including DEAE, Hydroxylapatite, Calcium Phosphate (see generally Current Protocols in Immunology, 1994, John Wiley & Sons, Inc., New York, N.Y.).

In another embodiment, bispecific molecules are purified by three-step successive affinity chromatography (Corvalan and Smith, 1987, Cancer Immunol. Immunother., 24:127-132): the first column is made of protein A bound to a solid matrix, wherein the Fc portion of the antibody binds protein A, and wherein the antibodies bind the column; followed by a second column that utilizes C3b-like receptor bound to a solid matrix which assays for C3b-like receptor binding via the anti-CR1 mAb portion of the bispecific molecule; and followed by a third column that utilizes specific binding of an antigenic molecule of interest which binds the antigen recognition portion of the bispecific molecule.

The bispecific molecules can also be purified by a combination of size exclusion HPLC and affinity chromatography. In one embodiment, the appropriate fraction eluted from size exclusion HPLC is further purified using a column containing an antigenic molecule specific to the antigen recognition portion of the bispecific molecule.

The bispecific molecules can be characterized by various methods known in the art. The yield of bispecific molecule can be characterized based on the protein concentration. In one embodiment, the protein concentration is determined using a lowry assay. Preferably, the bispecific molecule produced by the method of the present invention has a protein concentration of at least 0.100 mg/ml, more preferably at least 2.0 mg/ml, still more preferably at least 5.0 mg/ml, most preferably at least 10.0 mg/ml. In another embodiment, the concentration of the bispecific molecules is determined by measuring UV absorbance. The concentration is determined as the absorbance at 280 nm. Preferably, the bispecific molecule produced by the method of the present invention has an absorbance at 280 nm of at least 0.14.

The bispecific molecule of the invention can also be characterized using any other standard method known in the art. In one embodiment, high-performance size exclusion chromatography (HPLC-SEC) assay is used to determined the content of contamination by free IgG proteins. In preferred embodiments, the bispecific molecule composition produced by the method of the present invention has a contaminated IgG concentration of less than 6.0 mg/ml, more preferably less than 2.0 mg/ml, still more preferably less than 0.5 mg/ml, most preferably less than 0.03 mg/ml. In one embodiment, the bispecific molecules can be characterized by using SDS-PAGE to determine the molecular weight of the bispecific molecule.

The bispecific molecule can also be characterized based on the functional activity of the bispecific molecules. In one embodiment, the anti-CR1 binding activity is determined using ELISA with immobilized CR1 receptor molecules (attached to a solid phase, e.g., a microtiter plate) (see Porter et al., U.S. provisional application No. 60/380,211, which is incorporated herein by reference in its entirety). The assay is also referred to as a CR1/Antibody assay or CAA, and can be used generally to measure any anti-CR1 antibody, or HP or AHP containing an anti-CR1 antibody. In a preferred embodiment, ELISA/CR1 plates are prepared by incubating ELISA plates, e.g., high binding flat bottom ELISA plates (Costar EIA/RIA strip plate 2592) with a suitable amount of a bicarbonate solution of CR1 receptors. Preferably, the concentration of the bicarbonate solution of CR1 receptors is 0.2 ug/ml prepared from 5 mg/ml sCR1 receptors stock (Avant Technology Inc.) and a carbonate-bicarbonate buffer (pH 9.6, Sigma C-3041). In a preferred embodiment, 100 ul CR1 -bicarbonate solution is dispensed into each well of the ELISA plates and the plates are incubated at 4° C. overnight. The plates are then preferably washed using, e.g., a wash buffer (PBS, 0.1 % Tween-20, 0.05% 2-Chloroacetamide). In another preferred embodiment, a SuperBlock Blocking Buffer in PBS (Pierce) is added to the plates for about 30-60 min at room temperature after the wash. The plates can then be dried and stored at 4° C. The titration of anti-CR1 Abs or bispecific molecules can be carried out using a CR1 binding protein, e.g., human anti-CR1 IgG, as the calibrator. In a preferred embodiment, the calibrator a human anti-CR1 IgG having a concentration of 300 or 600 mg/ml. In one embodiment, the titration of the purified composition of bispecific molecules of the invention is carried out using PBS, 0.25% BSA, 0.1% Tween-20 as the diluent buffer, PBS, 0.1% Tween-20, 0.05% 2-Chloroacetamide as the wash buffer, TMB-Liquid Substrate System for ELISA (3,3′, 5.5′-Tetramethyl-Benzidine) and 2N H₂SO₄ as the stop solution. Preferably, the bispecific molecule composition produced by the method of the present invention has an CAA titer of at least 0.10 mg/ml, more preferably at least 0.20 mg/ml, still more preferably at least 0.30 mg/ml, and most preferably at least 0.50 mg/ml. In some embodiments, a specific anti-CR1 activity is determined. The specific anti-CR1 activity is a ratio of CAA and Lowry.

The antigen-binding activity can be determined using ELISA with immobilized antigen molecules.

In another embodiment, the bispecificity of a bispecific molecule comprising an antibody that binds a C3b-like receptor cross-linked with an antigen-binding antibody fragment that binds the protective antigen (PA) protein of Anthrax, i.e., specificities to CR-1 and PA is determined using ELISA assay. The assay is also referred to as HPCA assay. In a preferred embodiment, ELISA/CR1 plates are prepared as in CAA assay. Calibrators are bispecific molecule 14B7×7G9 (HC=1.0 g/ml, MC=0.5 μg/ml, LC=0.25 μg/ml). The HPCA assay can be carried out by the following protocol:

A. Binding bispecific molecule to CR-1 plate:
1. Dilute sample bispecific molecule to 5 μg/ml in ELISA diluent (1×PBS buffer, 0.25% BSA, 0.1% Tween 20, 0.05% 2-Chloroacetamide)
2. In a dilution plate, load samples at 5 μg/ml in rows A through H and serially dilute 1:3 fold (a maximum of four samples can be run on one plate). Run all samples, including calibrators in duplicates.
3. Transfer 100 μl of diluted samples from dilution plate into corresponding wells on CR-1 coated plate. Add 100 μl of HC, MC, and LC in duplicates to rows A11 and A12, B11 and B12, C11 and C12, respectively. Add 100 μl of diluent for blanks to five wells in duplicates.
4. Seal plate with the adhesive plate sealer and incubate at 37° C. for 1 hour.
5. Discard solution, wash plate with ELISA wash buffer (1×PBS, 0.1% Tween-20, 0.05% 2-Chloroacetamide) on auto plate washer with 5-cycle program.

B. Binding Biotin conjugated PA (b-PA) to bispecific molecule
1. Dilute b-PA to 2.5 ng/ml in ELISA diluent.
2. Transfer 100 μl of diluted b-PA into all wells (including blank wells).
3. Seal plate with adhesive plate sealer and incubate at 37° C. for 1 hour.
4. Discard solution, wash plate on auto plate washer with 5-cycle program.

C. Binding Horse radish Peroxidase-conjugated Streptavidin (SA-HRP, 0.5 mg/ml) to b-PA
1. Dilute SA-HRP 1: 10,000 in ELISA diluent.
2. Transfer 100 μl of diluted SA-HRP into all wells (including blank wells).
3. Seal plate with the adhesive plate sealer and incubate at 37° C. for 30 min.
4. Discard solution, wash plate on auto plate washer with 5-cycle program.

D. Signal development
1. Add 100 μl of pre-warmed TMB (Sigma, cat# T-0440) to all wells.
2. Incubate at room temperature for 15 min (protected from light).
3. Add 100 μl of stop solution (2N H₂SO₄), incubate at room temperature for 10 min.
4. Read plate at 450 nm using a plate reader.

The maximal absorbance value obtained, referred to as Max OD, can be used as a measure of the total activity of the bispecific molecule. In a preferred embodiment, Max OD is obtained from a 4-parameter sigmoidal fit of the optical density data. In another embodiment, a C₅₀ level is also determined. The C₅₀ is the concentration of a sample which yields 50% of the max OD.

### 5.3. Uses of Bispecific Molecules

The bispecific molecules of the present invention are useful in treating or preventing a disease or disorder associated with the presence of a pathogenic antigenic molecule. The pathogenic antigenic molecule can be any substance that is present in the circulation that is potentially injurious to or undesirable in the subject to be treated, including but are not limited to proteins or drugs or toxins, autoantibodies or autoantigens, or a molecule of any infectious agent or its products. A pathogenic antigenic molecule is any molecule containing an antigenic determinant (or otherwise capable of being bound by a binding domain) that is or is part of a substance (e.g., a pathogen) that is the cause of a disease or disorder or any other undesirable condition.

The preferred subject for administration of a bispecific molecule of the invention, for therapeutic or prophylactic purposes, is a mammal including but is not limited to non-human animals (e.g., horses, cows, pigs, dogs, cats, sheep, goats, mice, rats, etc.), and in a preferred embodiment, is a human or non-human primate.

Circulating pathogenic antigenic molecules cleared by the fixed tissue phagocytes include any antigenic moiety that is harmful to the subject. Examples of harmful pathogenic antigenic molecules include any pathogenic antigenic molecule associated with a parasite, fungus, protozoa, bacteria, or virus. Furthermore, circulating pathogenic antigenic molecules may also include toxins, immune complexes, autoantibodies, drugs, an overdose of a substance, such as a barbiturate, or anything that is present in the circulation and is undesirable or detrimental to the health of the host mammal. Failure of the immune system to effectively remove the pathogenic antigenic molecules from the mammalian circulation can lead to traumatic and hypovolemic shock (Altura and Hershey, 1968, Am. J. Physiol. 215:1414-9).

Moreover, non-pathogenic antigens, for example transplantation antigens, are mistakenly perceived to be harmful to the host and are attacked by the host immune system as if they were pathogenic antigenic molecules. The present invention further provides an embodiment for treating transplantation rejection comprising administering to a subject an effective amount of a bispecific molecule that will bind and remove immune cells or factors involved in transplantation rejection, e.g., transplantation antigen specific antibodies.

### 5.3.1 Autoimmune Antigens

In one embodiment, the pathogenic antigenic molecule to be cleared from the circulation includes autoimmune antigens. These antigens include but are not limited to autoantibodies or naturally occurring molecules associated with autoimmune diseases.

As one example, certain humans with hemophilia have been shown to be deficient in factor VIII. Recombinant factor VIII replacement treats this hemophilia. However, eventually some patients develop antibodies against factor VIII, thus interfering with the therapy. The bispecific molecules of the present invention prepared with an anti-anti-factor VIII antibody provides a therapeutic solution for this problem. In particular, a bispecific molecule with specificity of the first antigen recognition portion to a C3b-like receptor and specificity of the second antigen recognition portion to an anti-factor VIII autoantibody would be therapeutically useful in clearing the autoantibodies from the circulation, thus, ameliorating the disease.

Further examples of autoantibodies which can be cleared by the bispecific molecules of the present invention include, but are not limited to, autoantibodies to the following antigens: the muscle acetylcholine receptor (the antibodies are associated with the disease myasthenia gravis); cardiolipin (associated with the disease lupus); platelet associated proteins (associated with the disease idiopathic thrombocytopenic purpurea); the multiple antigens associated with Sjogren's Syndrome; the antigens implicated in the case of tissue transplantation autoimmune reactions; the antigens found on heart muscle (associated with the disease autoimmune myocarditis); the antigens associated with immune complex mediated kidney disease; the dsDNA and ssDNA antigens (associated with lupus nephritis); desmogleins and desmoplakins (associated with pemphigus and pemphigoid); or any other antigen which is characterized and is associated with disease pathogenesis.

When the above bispecific molecules are injected into the circulation of a human or non-human primate, the bispecific molecules will bind to red blood cells via the human or primate C3b receptor domain recognition site through their anti-CR1 antibody portions. The bispecific molecules will simultaneously associate with the autoimmune antigen, e.g., an autoantibody through their antigen-binding antibody fragments. The red blood cells which have the bispecific molecule/autoimmune antigen complex on their surface then facilitate the neutralization and clearance from the circulation of the bound pathogenic autoimmune antigen, e.g., an autoantibody.

In the present invention, the bispecific molecules facilitate pathogenic antigen or autoantibody binding to hematopoietic cells expressing a C3b-like receptor on their surface and subsequently clear the pathogenic antigen or autoantibody from the circulation, without also clearing the hematopoietic cells.

### 5.3.2 Infectious Diseases

In specific embodiments, infectious diseases are treated or prevented by administration of a bispecific molecule that binds both an antigen of an infectious disease agent and a C3b-like receptor. Thus, in such an embodiment, the pathogenic antigenic molecule is an antigen of an infectious disease agent.

Such antigen can be but is not limited to: influenza virus hemagglutinin (Genbank accession no. J02132; Air, 1981, Proc. Natl. Acad. Sci. USA 78:7639-7643; Newton et al., 1983, Virology 128:495-501), human respiratory syncytial virus G glycoprotein (Genbank accession no. Z33429; Garcia et al., 1994, J. Virol.; Collins et al., 1984, Proc. Natl. Acad. Sci. USA 81:7683), core protein, matrix protein or other protein of Dengue virus (Genbank accession no. M19197; Hahn et al., 1988, Virology 162:167-180), measles virus hemagglutinin (Genbank accession no. M81899; Rota et al., 1992, Virology 188:135-142), herpes simplex virus type 2 glycoprotein gB (Genbank accession no. M14923; Bzik et al., 1986, Virology 155:322-333), poliovirus I VP1 (Emini et al., 1983, Nature 304:699), envelope glycoproteins of HIV I (Putney et al., 1986, Science 234:1392-1395), hepatitis B surface antigen (Itoh et al., 1986, Nature 308:19; Neurath et al., 1986, Vaccine 4:34), diphtheria toxin (Audibert et al., 1981, Nature 289:543), streptococcus 24M epitope (Beachey, 1985, Adv. Exp. Med. Biol. 185:193), gonococcal pilin (Rothbard and Schoolnik, 1985, Adv. Exp. Med. Biol. 185:247), pseudorabies virus g50 (gpD), pseudorabies virus II (gpB), pseudorabies virus gIII (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydodysenteriae protective antigen, bovine viral diarrhea glycoprotein 55, Newcastle disease virus hemagglutinin-neuraminidase, swine flu hemagglutinin, swine flu neuraminidase, foot and mouth disease virus, hog colera virus, swine influenza virus, African swine fever virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein I), a glycoprotein of La Crosse virus (Gonzales-Scarano et al., 1982, Virology 120:42), neonatal calf diarrhea virus (Matsuno and Inouye, 1983, Infection and Immunity 39:155), Venezuelan equine encephalomyelitis virus (Mathews and Roehrig, 1982, J. Immunol. 129:2763), punta toro virus (Dalrymple et al., 1981, Replication of Negative Strand Viruses, Bishop and Compans (eds.), Elsevier, N.Y., p. 167), murine leukemia virus (Steeves et al., 1974, J. Virol. 14:187), mouse mammary tumor virus (Massey and Schochetman, 1981, Virology 115:20), hepatitis B virus core protein and/or hepatitis B virus surface antigen or a fragment or derivative thereof (see, e.g., U.K. Patent Publication No. GB 2034323A published Jun. 4, 1980; Ganem and Varmus, 1987, Ann. Rev. Biochem. 56:651-693; Tiollais et al., 1985, Nature 317:489-495), of equine influenza virus or equine herpesvirus (e.g., equine influenza virus type A/Alaska 91 neuraminidase, equine influenza virus type A/Miami 63 neuraminidase, equine influenza virus type A/Kentucky 81 neuraminidase equine herpesvirus type 1 glycoprotein B, and equine herpesvirus type 1 glycoprotein D, antigen of bovine respiratory syncytial virus or bovine parainfluenza virus (e.g., bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and the bovine parainfluenza virus type 3 hemagglutinin neuraminidase), bovine viral diarrhea virus glycoprotein 48 or glycoprotein 53.

Additional diseases or disorders that can be treated or prevented by the use of a bispecific molecule of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, hantavirus, coxsackie virus, mumps virus, measles virus, rubella virus, polio virus, human immunodeficiency virus type I (HIV-I), and human immunodeficiency virus type II (HIV-II), any picomaviridae, enteroviruses, caliciviridae, any of the Norwalk group of viruses, togaviruses, such as Dengue virus, alphaviruses, flaviviruses, coronaviruses, rabies virus, Marburg viruses, ebola viruses, parainfluenza virus, orthomyxoviruses, bunyaviruses, arenaviruses, reoviruses, rotaviruses, orbiviruses, human T cell leukemia virus type I, human T cell leukemia virus type II, simian immunodeficiency virus, lentiviruses, polyomaviruses, parvoviruses, Epstein-Barr virus, human herpesvirus-6, cercopithecine herpes virus 1 (B virus), and poxviruses.

Bacterial diseases or disorders that can be treated or prevented by the use of bispecific molecules of the present invention include, but are not limited to, Mycobacteria rickettsia, Mycoplasma, Neisseria spp. (e.g., Neisseria menigitidis and Neisseria gonorrhoeae), Legionella, Vibrio cholerae, Streptococci, such as Streptococcus pneumoniae, Corynebacteria diphtheriae, Clostridium tetani, Bordetella pertussis, Haemophilus spp. (e.g., influenzae), Chlamydia spp., enterotoxigenic Escherichia coli, Streptococcus B, Staphylococcus, Yersinia pestis (plague), Francisella tularensis, and Bacillus anthracis (anthrax), etc.

Protozoal diseases or disorders that can be treated or prevented by the use of bispecific molecules of the present invention include, but are not limited to, plasmodia, eimeria, Leishmania, and trypanosoma.

In a specific embodiment, the invention provides a method and compositions for treating Anthrax infection. The method comprises administrating to a patient a therapeutical sufficient amount of a bispecific molecule comprising an antibody that binds a C3b-like receptor cross-linked with an antigen-binding antibody fragment which binds the protective antigen (PA) protein of Bacillus anthracis (Anthrax), a common component of the lethal and edema toxins of Anthrax (see, e.g., Little et al., 1991, Biochem Biophys Res Commun.180:531-7; Little et al., 1988, Infect Immun. 56:1807-13). The protective antigen protein of Anthrax was shown to be required for toxicity (Little et al., 1988, Infect Immun. 56:1807-13). The bispecific molecules can be used to remove PA from the circulation thereby ameliorating the toxic effect of Anthrax. In one embodiment, the antibody fragment is the Fab fragment of an antibody 14B7 which binds PA (see, e.g., Little et al., 1991, Biochem Biophys Res Commun.180:531-7; Little et al., 1988, Infect Immun. 56:1807-13). In another embodiment, the antibody fragment is a single-chain antibody derived from 14B7 (14B7scAb). The 14B7scAb consists of a single chain Fv of 14B7 fused with a human constant k domain (see, e.g., Maynard et al., Nature Biotechnology 20:597-601). In a preferred embodiment, the antibody that binds a C3b-like receptor is the murine anti-CR1 IgG 7G9. In a preferred embodiment, the bispecific molecule is produced by cross-linking an anti-CR1 mAb, e.g., 7G9, and an anti-PA Fab fragment, e.g., 14B7Fab, using N-succinimidyl-S-acetyl-thioacetate (SATA) and sulfosuccinimidyl 4-(N-maleimidomethyl) cyclohexane-1-carboxylate (sSMCC) as the cross-linking agents. In another preferred embodiment, the bispecific molecule is produced by cross-linking an anti-CR1 mAb, e.g., 7G9, and an anti-PA single chain antibody, e.g., 14B7scAb, using N-succinimidyl-S-acetyl-thioacetate (SATA) and NHS-poly(ethylene glycol)-maleimide (PEG-MAL) as the cross-linking agents. In still another preferred embodiment, the bispecific molecule is produced by cross-linking an anti-CR1 mAb, e.g., 7G9, and an anti-PA single chain antibody, e.g., 14B7Fab, using N-succinimidyl-S-acetyl-thioacetate (SATA) and NHS-poly(ethylene glycol)-maleimide (PEG-MAL) as the cross-linking agents.

### 5.3.3 Additional Pathogenic Antigenic Molecules

In one embodiment, the pathogenic antigenic molecule to be cleared from the circulation by the methods and compositions of the present invention encompass any serun drug, including but is not limited to barbiturates, tricyclic antidepressants, and Digitalis.

In another embodiment, the pathogenic antigenic molecule to be cleared includes any serum antigen that is present as an overdose and can result in temporary or permanent impairment or harm to the subject. This embodiment particularly relates to drug overdoses.

In another embodiment, the pathogenic antigenic molecule to be cleared from the circulation include naturally occurring substances. Examples of naturally occurring pathogenic antigenic molecules that could be removed by the methods and compositions of the present invention include but are not limited to low density lipoproteins, interleukins or other immune modulating chemicals and hormones.

### 5.3.4. Cocktails of Bispecific Molecules

Various purified bispecific molecules can be combined into a “cocktail” of bispecific molecules. Such cocktail of bispecific molecules can include bispecific molecules each having an anti-CR1 mAb conjugated to any one of several desired antigen-binding antibody fragments. For example, the bispecific molecule cocktail comprises a plurality of different bispecific molecules, wherein each different bispecific molecule in the plurality contains a different antigen-binding antibody fragment that targets a different pathogens. Such bispecific molecule cocktails are useful as personalized medicine tailored according to the need of individual patients. Alternatively, a cocktail of bispecific molecules can include bispecific molecules each having a different anti-CR1 mAb which binds a different sites on a CR1 receptor conjugated to a desired antigen-binding antibody fragment. Such bispecific molecule cocktails can be used to increase the number of pathogens bound to each red blood cell by utilizing different CR1 binding sites.

### 5.3.5. Dose of Bispecific Molecules

The dose can be determined by a physician upon conducting routine tests. Prior to administration to humans, the efficacy is preferably shown in animal models. Any animal model for a blood bome disease known in the art can be used.

More particularly, the dose of the bispecific molecule can be determined based on the hematopoietic cell concentration and the number of C3b-like receptor epitope sites bound by the anti-C3b-like receptor monoclonal antibodies per hematopoietic cell. If the bispecific molecule is added in excess, a fraction of the bispecific molecule will not bind to hematopoietic cells, and will inhibit the binding of pathogenic antigens to the hematopoietic cell. The reason is that when the free bispecific molecule is in solution, it will compete for available pathogenic antigen with bispecific molecule bound to hematopoietic cells. Thus, the bispecific molecule-mediated binding of the pathogenic antigens to hematopoietic cells follows a bell-shaped curve when binding is examined as a function of the concentration of the input bispecific molecule concentration.

Viremia may result in up to 10⁸-10⁹ viral particles/ml of blood (HIV is 10⁶/ml; (Ho, 1997, J. Clin. Invest. 99:2565-2567)); the dose of therapeutic bispecific molecules should preferably be, at a minimum, approximately 10 times the antigen number in the blood.

In general, for antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration are often possible.

As defined herein, a therapeutically effective amount of bispecific molecule (i.e., an effective dosage) ranges from about 0.001 to 30 mg/kg body weight, preferably about 0.01 to 25 mg/kg body weight, more preferably about 0.1 to 20 mg/kg body weight, and even more preferably about 0.1 to 10 mg/kg body weight.

The skilled artisan will appreciate that certain factors may influence the dosage required to effectively treat a subject, including but is not limited to the severity of the disease or disorder, previous treatments, the general health and/or age of the subject, and other diseases present. Moreover, treatment of a subject with a therapeutically effective amount of a bispecific molecule can include a single treatment or, preferably, can include a series of treatments. In a preferred example, a subject is treated with a bispecific molecule in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a bispecific molecule, used for treatment may increase or decrease over the course of a particular treatment. Changes in dosage may result and become apparent from the results of diagnostic assays as described herein.

It is understood that appropriate doses of bispecific molecule agents depends upon a number of factors within the ken of the ordinarily skilled physician, veterinarian, or researcher. The dose(s) of the bispecific molecule will vary, for example, depending upon the identity, size, and condition of the subject or sample being treated, further depending upon the route by which the composition is to be administered, if applicable, and the effect which the practitioner desires the bispecific molecule to have upon a pathogenic antigenic molecule or autoantibody.

It is also understood that appropriate doses of bispecific molecules depend upon the potency of the bispecific molecule with respect to the antigen to be cleared. Such appropriate doses may be determined using the assays described herein. When one or more of these bispecific molecules is to be administered to an animal (e.g., a human) in order to clear an antigen, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the bispecific molecule employed, the age, body weight, general health, gender, and diet of the subject, the time of administration, the route of administration, the rate of excretion, any drug combination, and the concentration of antigen to be cleared.

### 5.3.6. Pharmaceutical Formultion and Administration

The bispecific molecules of the invention can be incorporated into pharmaceutical compositions suitable for administration. Such compositions typically comprise bispecific molecule and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the bispecific molecule, use thereof in the compositions is contemplated. Supplementary bispecific molecules can also be incorporated into the compositions.

A pharmaceutical composition of the invention is formulated to be compatible with its intended route of administration. The preferred route of administration is intravenous. Other examples of routes of administration include parenteral, intradermal, subcutaneous, transdermal (topical), and transmucosal. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that the viscosity is low and the bispecific molecule is injectable. It must be stable under the conditions of manufacture and storage and must be preserved against the contaminating action of microorganisms such as bacteria and fungi.

The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the bispecific molecule (e.g., one or more bispecific molecules) in the required amount in an appropriate solvent with one or a combination of ingredients enumerated above, as required, followed by filtered sterilization. Generally, dispersions are prepared by incorporating the bispecific molecule into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof

In one embodiment, the bispecific molecules are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 which is incorporated herein by reference in its entirety.

It is advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of bispecific molecule calculated to produce the desired therapeutic effect in association with the required pharmaceutical carrier. The specification for the dosage unit forms of the invention are dictated by and directly dependent on the unique characteristics of the bispecific molecule and the particular therapeutic effect to be achieved, and the limitations inherent in the art of compounding such a bispecific molecule for the treatment of individuals.

The pharmaceutical compositions can be included in a kit, in a container, pack, or dispenser together with instructions for administration.

### 5.3.7. Ex Vivo Preparation of the Bispecific Molecule

In an alternative embodiment, the bispecific molecule, such as a bispecific molecule, is prebound to hematopoietic cells of the subject ex vivo, prior to administration. For example, hematopoietic cells are collected from the individual to be treated (or alternatively hematopoietic cells from a non-autologous donor of the compatible blood type are collected) and incubated with an appropriate dose of the therapeutic bispecific molecule for a sufficient time so as to allow the antibody to bind the C3b-like receptor on the surface of the hematopoietic cells. The hematopoietic cell/bispecific molecule mixture is then administered to the subject to be treated in an appropriate dose (see, for example, Taylor et al., U.S. Pat. No. 5,487,890).

The hematopoietic cells are preferably blood cells, most preferably red blood cells.

Accordingly, in a specific embodiment, the invention provides a method of treating a mammal having an undesirable condition associated with the presence of a pathogenic antigenic molecule, comprising the step of administering a hematopoietic cell/bispecific molecule complex to the subject in a therapeutically effective amount, said complex consisting essentially of a hematopoietic cell expressing a C3b-like receptor bound to one or more bispecific molecules. The method alternatively comprises a method of treating a mammal having an undesirable condition associated with the presence of a pathogenic antigenic molecule comprising the steps of (a) contacting a bispecific molecule with hematopoietic cells expressing a C3b-like receptor, to form a hematopoietic cell/bispecific molecule complex; and (b) administering the hematopoietic cell/bispecific molecule complex to the mammal in a therapeutically effective amount.

The invention also provides a method of making a hematopoietic cell/bispecific molecule complex comprising contacting a bispecific molecule with hematopoietic cells that express a C3b-like receptor under conditions conducive to binding, such that a complex forms, said complex consisting essentially of a hematopoietic cell bound to one or more bispecific molecules.

Taylor et al. (U.S. Pat. No. 5,879,679, hereinafter “the '679 patent”) have demonstrated in some instances that the system saturates because the concentration of autoantibodies (or other pathogenic antigen) in the plasma is so high that even at the optimum input of bispecific molecules, not all of the autoantibodies can be bound to the hematopoietic cells under standard conditions. For example, for a very high titer of autoantibody sera, a fraction of the autoantibody is not bound to the hematopoietic cells due to its high concentration.

However, saturation can be solved by using combinations of bispecific molecules which contain monoclonal antibodies that bind to different sites on a C3b-like receptor. For example, the monoclonal antibodies 7G9 and 1B4 bind to separate and non-competing sites on the primate C3b receptor. Therefore, a “cocktail” containing a mixture of two bispecific molecules, each made with a different monoclonal antibody to the C3b-like receptor, may give rise to greater binding of bispecific molecules to red blood cells. The bispecific molecules of the present invention can also be used in combination with certain fluids used for intravenous infusions.

In yet another embodiment, the bispecific molecule, such as a bispecific molecule, is prebound to red blood cells in vitro as described above, using a blend of at least two different bispecific molecules. In this embodiment, the two different bispecific molecules bind to the same antigen, but also bind to distinct and non-overlapping recognition sites on the C3b-like receptor. By using at least two non-overlapping bispecific molecules for binding to the C3b-like receptor, the number of bispecific molecule-antigen complexes that can bind to a single red blood cell is increased. Thus, by allowing more than one bispecific molecule to bind to a single C3b-like receptor, antigen clearance is enhanced, particularly in cases where the antigen is in very high concentrations (see for example the '679 patent, column 6, lines 41-64).

### 5.4. Kits

The invention also provides kits containing the bispecific molecules of the invention. Kits containing the pharmaceutical compositions of the invention are also provided.

### 6. EXAMPLES

The following examples describe the production of bispecific molecules comprising an anti-CR1 mAb and an antibody fragment that binds the protective antigen (PA) protein of Bacillus anthracis (Anthrax), a common component of the lethal and edema toxins of Anthrax (see, e.g., Little et al., 1991, Biochem Biophys Res Commun.180:531-7; Little et al., 1988, Infect Immun. 56:1807-13). It was shown that binding of PA to cell receptors is required for toxicity (see, e.g., Little et al., 1988, Infect Immun. 56:1807-13). The antibody fragments are the Fab fragment of an antibody 14B7 which binds PA (see, e.g., Little et al., 1991, Biochem Biophys Res Commun.180:531-7; Little et al., 1988, Infect Immun. 56:1807-13) and a single chain antibody fragment consisting of a single chain Fv of murine monoclonal antibody 14B7 fused with a human constant k domain (see, e.g., Maynard et al., Nature Biotechnology 20:597-601). The bispecific molecules produced in the Examples can therefore be used for treatment of Anthrax infection by removing PA from the circulation. Example 6.1 describes the production of bispecific molecules comprising an anti-CR1 mAb, 7G9, and an anti-PA Fab fragment, 14B7Fab, using N-succinimidyl-S-acetyl-thioacetate (SATA) and sulfosuccinimidyl 4-(N-maleimidomethyl) cyclohexane-1-carboxylate (sSMCC) as the cross-linking agents; Example 6.2 describes the production of bispecific molecules comprising 7G9 and an anti-PA single chain antibody, 14B7scAb, using N-succinimidyl-S-acetyl-thioacetate (SATA) and NHS-poly(ethylene glycol)-maleimide (PEG-MAL) as the cross-linking agents; and Example 6.3 describes the production of bispecific molecules comprising 7G9 and 14B7Fab using N-succinimidyl-S-acetyl-thioacetate (SATA) and NHS-poly(ethylene glycol)-maleimide (PEG-MAL) as the cross-linking agents.

### 6.1. Bispecific Molecules: 7G9-SMCC-14B7Fab

A hybridoma cell line secreting a high-affinity anti-CR1 monoclonal antibody was used to produce the 7G9 (murine IgG₂a, kappa) anti-CR1 mAb. A master cell bank (MCB) was generated from this cell line and tested (Charles River Tektagen) for mouse antibody production, mycoplasma and sterility. The 7G9 antibody used in the production of the bispecific molecules was produced and purified from ascites fluid.

The anthrax PA binding antibody fragment was the Fab fragment of the anthrax PA binding mAb 14B7. The Fab fragment was produced by digesting the 14B7 mAb using papain.

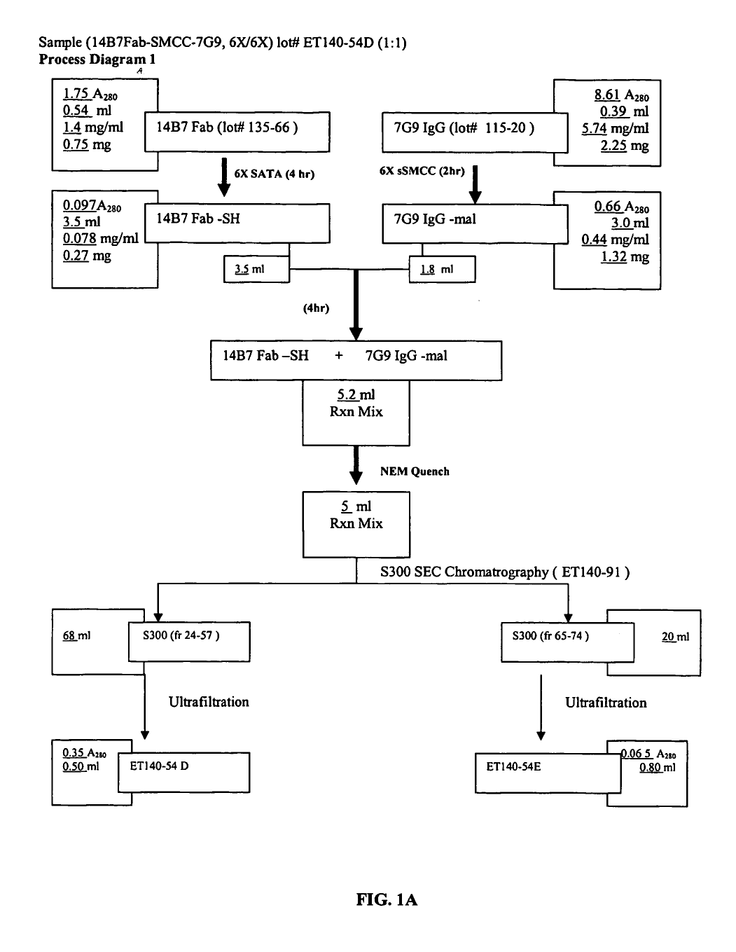

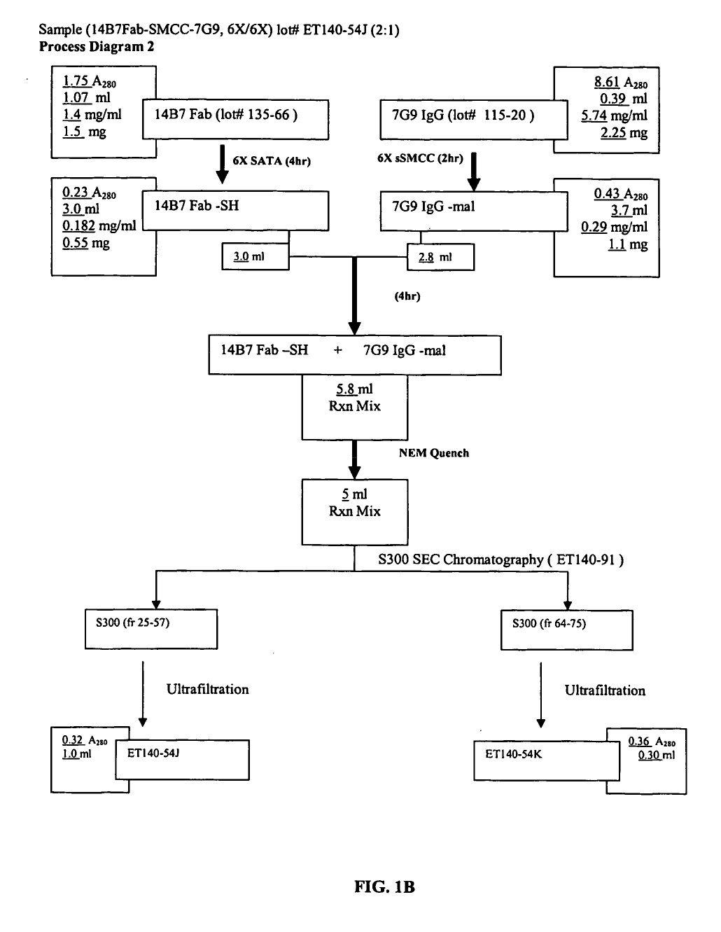

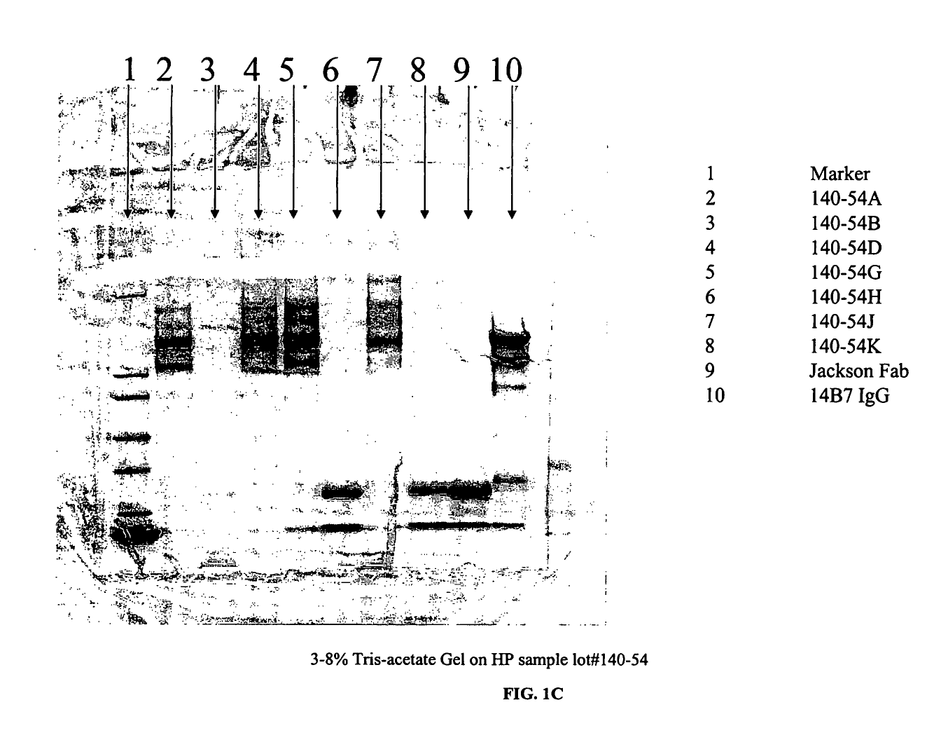

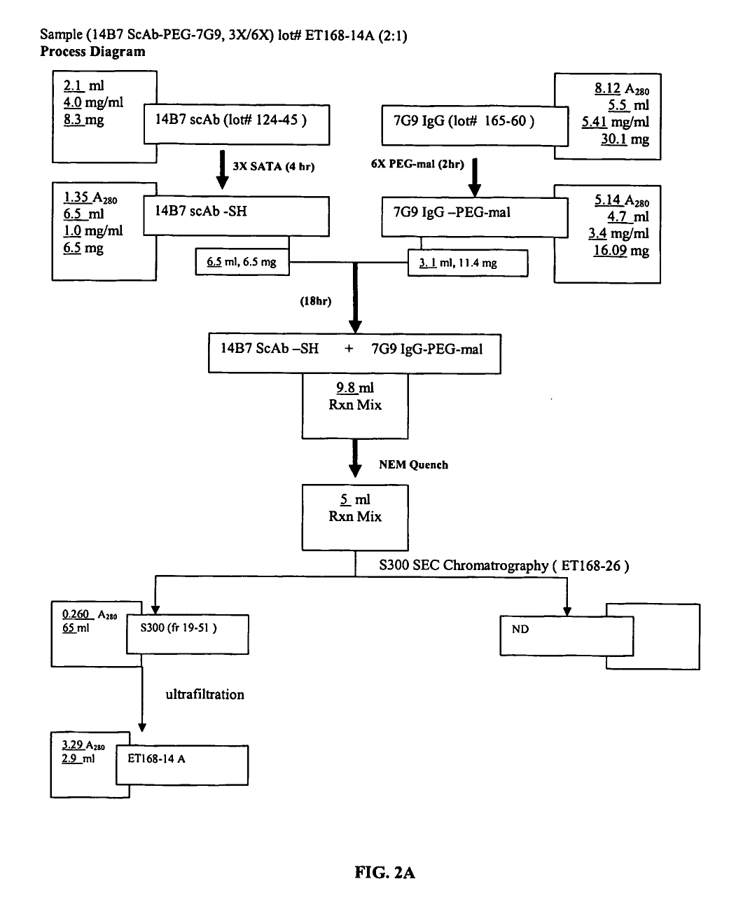

Flow charts showing the production processes are depicted in FIGS. 1A and 1B.

The 14B7Fab antigen-binding antibody fragment was derivatized with SATA as follows. A solution of SATA in DMSO was prepared. The 14B7 Fab was dialyzed against PBSE buffer overnight in a refrigerator. 7.2 ul of SATA solution (0.025 mg, 108 nmol) was added to 18 nmol of dialyzed 14B7 Fab (at a molar ratio of about 6:1). The reactants was incubated at room temperature for about 2 hours with gentle inversion every 15-30 min. A hydroxylamine HCl solution was prepared by adding 0.76 g hydroxyamine and 1.0 ml 0.5M EDTA to 25 ml MES at pH7.5. 72 ul of the Hydroxylamine HCl solution (2.79 mg, 36 umol) was added to the reaction mixture from the SATA coupling step (molar ratio of about 2000:1 to 14B7Fab) and incubated for about 2 hours at room temperature under argon atmosphere. The reaction mixture was then desalted by chromatography using an Amersham Hi-Prep desalting column (26/10) in MES buffer. 3.8 ml of pooled sample was recovered. The recovered sample was 2.2 mg, and had a protein concentration of 0.57 mg/ml (A280), about 81.4% recovery. The SATA modified antigen binding antibody fragment 14B7fab-SH was eluted in the void volume with PBSE buffer.

The 7G9 antibody was derivatized with sSMCC as follows: a fresh stock solution of 6× sSMCC conjugation solution was prepared in PBSE buffer; the antibody was dialyzed exhaustively against PBSE buffer; the coupling reaction was initiated by combining the antibody and sSMCC at a molar ratio of 1:6; the reactants are mixed by inversion and incubated at room temperature for 2 hours with mixing; and the sSMCC-antibody was recovered by size exclusion chromatography using FPLC with two Pharmacia 26/10 Desalting Columns in series (cat#17-5087-01). The column was pre-washed with distilled water followed by PBSE buffer according to the manufacturer's instructions before loaded with the reaction mixture. The maleimide modified antibody 7G9-MAL was eluted in the void volume with PBSE buffer.

Two different 14B7Fab-SH and 7G9-MAL conjugation reaction mixtures, designated as ET140-90 and ET140-91, respectively, were prepared. ET140-90 combined 14B7Fab-SH and 7G9-MAL at a molar ratio of 1:1 (14B7Fab-SH:7G9-MAL), whereas ET140-91 at a molar ratio of 2:1. The reaction mixtures were incubated for 4 hours. ET140-90 and ET140-91 were left for 6 and 7 days, respectively. ET140-90 and ET140-91 were quenched in N-Ethylmaleimide (NEM, Pierce, No 23030, CAS 128-53-0) and fractioned using S300 SEC chromatography.

Sample ET140-54D was pooled fractions from the S300 column run of the reaction mixture ET140-90. The S300 column run (ET140-90), loaded with 5-ml reaction mixture, generated 108, 2-ml fractions. A 68-ml pool from fractions 24 through 57 was labeled as ET140-54D. Sample D was further processed by ultrafiltration to concentrate the preparations to a final volume of 0.5 ml. SDS-PAGE analysis shows that sample D contains free antibodies and higher MW bispecific molecules.

Sample ET140-54J was pooled fractions from the S300 column run of the reaction mixture ET140-91. The S300 column run (ET140-91), loaded with 5-ml reaction mixture, generated 108, 2-ml fractions. A pool from fractions 25 through 57 was labeled as ET140-54J. The pooling volumes were not recorded. Sample J were further processed by ultrafiltration to concentrate the preparations to a final volume of 1.0 ml. SDS-PAGE analysis shows that sample J contains free antibodies and the higher MW bispecific molecules. FIG. 1C shows a photograph of a Tris-Glycine SDS PAGE containing the sample ET140-54J.

SDS-PAGE, functional CR1 binding (CAA), functional PA binding (PAA), bivalency binding (HPCA) and protein content (Lowry) data for samples ET140-54 J and ET140-54D are summarized in Table I.

Lowry data show that 0.125 milligrams of protein was recovered in the bispecific molecule fraction, 140-54D. This represents a 6% of the total starting input antibody (2.1 milligrams). SDS-PAGE analysis shows that sample D contained multiple conjugated species and approximately 50% unreacted antibodies.

Lowry data show that 0.247 milligrams of protein was recovered in the bispecific molecule fraction, 140-54J. This represents a 8% of the total starting input antibody (3.2 milligrams). SDS-PAGE analysis shows that sample J contained multiple conjugated species and approximately 50% unreacted antibodies.

Both fractions, 140-54J and 140-54D, demonstrated similar CR1 binding activity as indicated by the CAA assay. Both fractions, 140-54J and 140-54D, demonstrated anthrax PA binding activity as indicated by the PAA assay. Both fractions, 140-54J and 140-54D, demonstrated similar bivalent binding activity indicating successful cross-linking of the two functional components, as indicated by the HPCA assay.

TABLE I
Characterization of ET140-54D and ET140-54J

| | ET140-54D | ET140-54J |
|---|---|---|
| A280 (mg/ml) | 235 | 214.3 |
| Lowry (mg/ml) | 250.4 | 247 |
| CAA (mg/ml) | 151.6 | 152.6 |
| CAA/Lowry | 0.6 | 0.6 |
| HPCA C₅₀ value (mg/ml) | 0.253 | 0.208 |
| Max OD | 0.788 | 1.184 |
| PAA (mg/ml) | 11.90 | 18.71 |

### 6.2. Bispecific Molecules: 7G9-PEG-14B7scAb

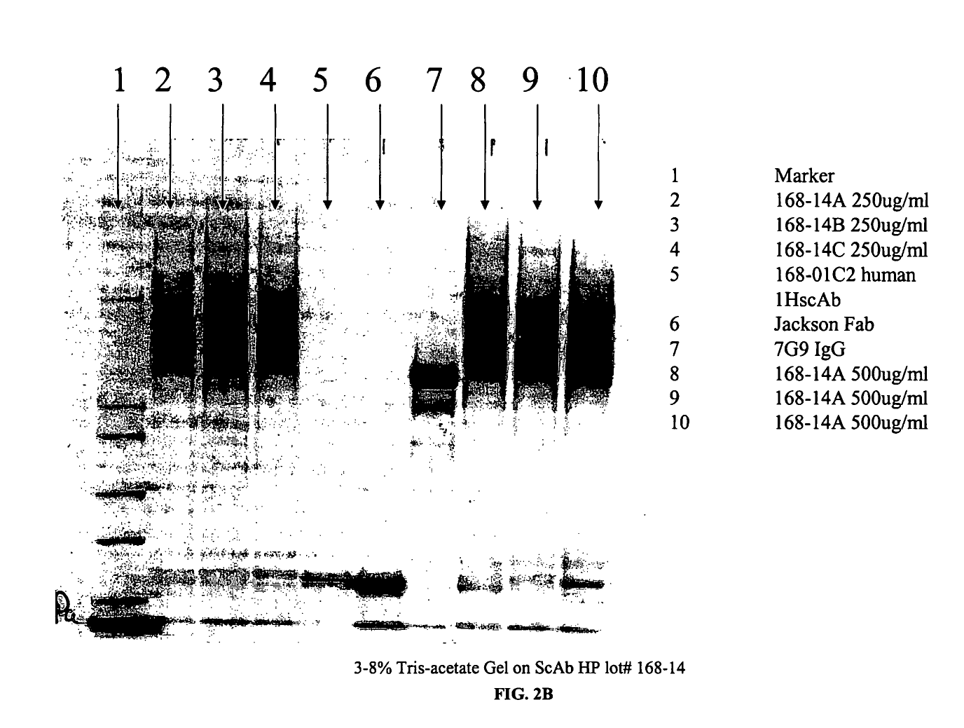

In this example, the anthrax PA binding antibody fragment was a single chain antibody fragment consisting of a single chain Fv of murine monoclonal antibody 14B7 fused with a human constant k domain The scAb fragment was prepared according to the procedure described in Maynard et al., Nature Biotechnology 20:597-601. A flow chart showing the production process is depicted in FIG. 2A.

The 14B7scAb antigen-binding antibody fragment was derivatized with SATA as described in Example 6.1. 14B7scAb was derivatized using a molar ratios of 1:3 (14B7scAb:SATA).

The 7G9 antibody was derivatized with NHS-PEG-MAL (Shearwater Polymers, Cat. # 2D2Z0F021) as follows. A 50 mg/ml MES solution of NHS-PEG-MAL (14.7 nmol/ul) was prepared. 7.34 ul of the NHS-PEG-MAL solution was added to 1.5 ml 7G9 (36 nmol) (molar ratio of about 3:1 PEG:antibody). The reactants was incubated at room temperature for about 2 hours with gentle inversion every 15-30 min. The reaction mixture is then desalted by chromatography using an Amersham Hi-Prep desalting column in MES buffer. The reaction mixture was then desalted by chromatography using an Amersham Hi-Prep desalting column (26/10) in MES buffer. 3.3 ml of pooled sample was recovered. The recovered sample was 1.5 mg, and had a protein concentration of 0.45 mg/ml (A280), representing a 3.3% recovery. The PEG-MAL modified antibody 7G9-PEG-MAL was eluted in the void volume with PBSE buffer.

A reaction mixture of 14B7scAb-SH and 7G9-PEG-MAL with a molar ratio of 2:1 (14B7Fab-SH:7G9-PEG-MAL) was prepared. The reaction mixtures were incubated for 18 hours. The mixture was quenched in NEM and fractioned using S300 SEC chromatography the next day.

Sample ET168-14A was a pool of fractions from an S300 column run. The S300 column run (ET168-26 ), loaded with 5-ml concentrated reaction mixture, generated 120, 2-ml fractions. A 65-ml pool from fractions 19 through 51 was labeled as ET168-14A. The pooling process was recorded on ET168-26. Sample ET168-14A was further processed by ultrafiltration to concentrate the product mixture to a final volume of 2.9 ml. SDS-PAGE analysis shows sample ET168-14A contains 10% free scAb, 45% monomer (PEG-7G9) and 45% higher MW bispecific molecules. FIG. 2B shows a photograph of a Tris-Glycine SDS PAGE containing the sample ET168-14A.

SDS-PAGE, functional CR1 binding (CAA), functional PA binding (PAA), bivalency binding (HPCA) and protein content (Lowry) data for samples ET168-14A are summarized in Table II.

Lowry data show that 9.3 milligrams of protein was recovered in the final bispecific molecule mixture, 168-14A. This represents a 32 % of the total starting input antibody (28 milligrams). SDS-PAGE analysis shows sample 168-14A contained multiple conjugated species and approximately 45% non-cross linked antibodies. SDS-gel shows conjugate size of approximately 200 kD. At 200 kD expected molar ratio of 1:1 (ScAb:7G9).

Sample ET168-14A had CR1 binding activity as indicated by the CAA assay. Specific activity was calculated at 0.58.

The sample ET168-14A demonstrated anthrax PA binding activity as indicated by the PAA assay. Specific activity was calculated 0.18 and the comparison to reference 14B7 antibody indicated approximately (0.18/0.71) 25% of the activity of an unmodified antibody. Specific activity of unmodified scab is not recorded.

The sample, ET168-14A, demonstrated bivalent binding activity indicating successful crosslinking of the two functional components, as indicated by the HPCA assay.

TABLE II
Characterization of ET168-14A

| | ET168-14A |
|---|---|
| HPCA C₅₀ value (mg/ml) | 0.166 |
| Max OD | 2.895 |

### 6.3. Bispecific Molecules: 7G9-PEG-14B7Fab

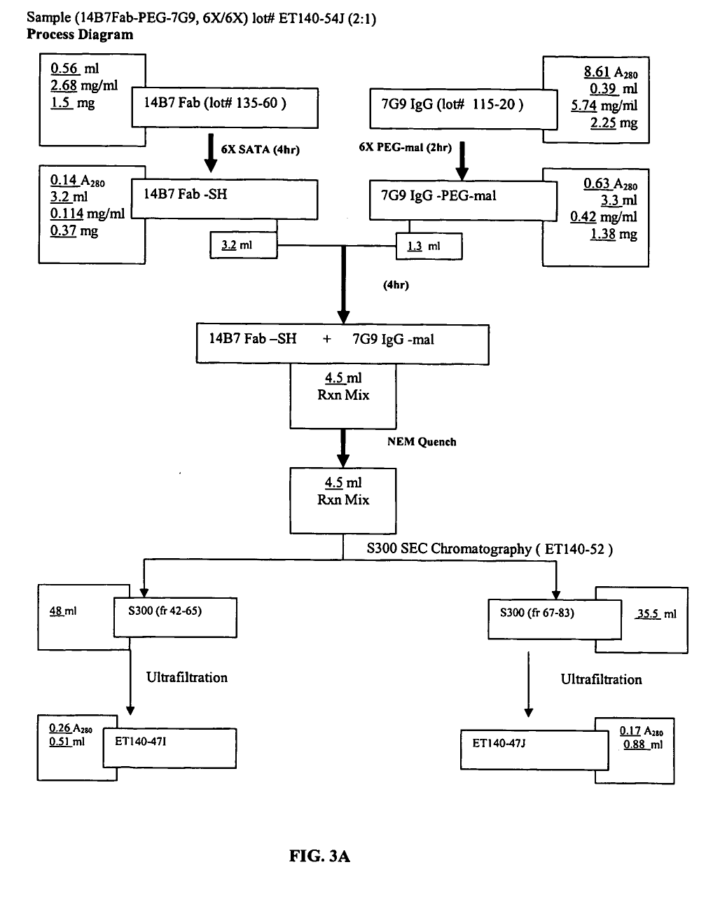

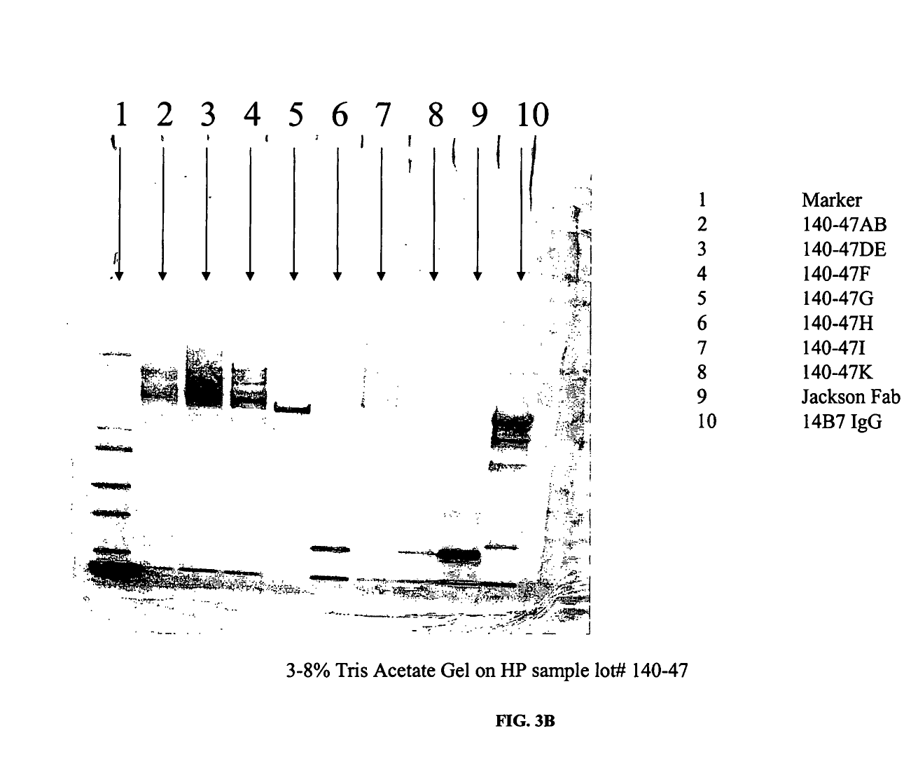

In this example, the production of bispecific molecule 7G9-PEG-14B7Fab is described. A flow chart showing the production process is depicted in FIG. 3A.

The 14B7Fab antigen-binding antibody fragment was derivatized using SATA as described in Example 6.1. The 7G9 antibody was derivatized with NHS-PEG-MAL as described in Example 6.2.

A reaction mixture of 14B7scAb-SH and 7G9-PEG-MAL with a molar ratio of 2:1 (14B7Fab-SH:7G9-PEG-MAL) was prepared. The reaction mixtures were incubated for 4 hours. The mixture was quenched in NEM and fractioned using S300 SEC chromatography after two days.

Sample ET140-47I was pooled fractions from the S300 column run of the reaction mixture. The S300 column run, loaded with 4.5-ml reaction mixture, generated 140, 2-ml fractions. A 68-ml pool from fractions 24 through 57 was labeled ET140-54D. A 65-ml pool from fractions 42-64 was labeled ET140-47I. Sample ET140-47I was further processed by ultrafiltration to concentrate the preparations to a final volume of 0.5 ml. SDS-PAGE analysis showed that sample D contains free antibodies and higher MW bispecific molecules. FIG. 3B shows a photograph of a Tris-Glycine SDS PAGE containing the sample ETI40-47I.

SDS-PAGE, functional CR1binding (CAA), functional PA binding (PAA), bivalency binding (HPCA) and protein content (Lowry) data for samples ET140-471 are summarized in Table III.

Lowry data showed that 0.070 milligrams of protein was recovered in the bispecific molecule fraction, 140-471. This represents a 3% of the total starting input antibody (2.4 milligrams). SDS-PAGE analysis showed that sample D contained multiple conjugated species and approximately 50% unreacted antibodies.

Sample ET140-47I had CR1 binding activity as indicated by the CAA assay. Specific activity was calculated at 0.33 and the comparison to reference 7G9 antibody indicated approximately 39% (0.33/0.85) of the unmodified antibody activity.

Sample ET140-47I demonstrated anthrax PA binding activity as indicated by the PAA assay. Specific activity was calculated 0.07. Specific activity of unmodified 14B7 was not recorded.

Sample ET140-47I demonstrated bivalent binding activity indicating successful crosslinking of the two functional components, as indicated by the HPCA assay.

TABLE III
Characterization of ET140-47I

| | ET140-47I |
|---|---|
| HPCA C₅₀ value (mg/ml) | 0.217 |
| Max OD | 1.419 |

### 6.4. A 14B7scAb-7G9 Heteropolymer Provides Long Period of Protection Against Anthrax Toxin in Rat

This example shows that a bispecific 14B7scAb-7G9 (scAbHP) not only retained binding activity of the 14B7scAb, but also showed desirable biological properties such as long term stability in the blood stream.

The 7G9 antibody was derivatized with SATA as follows: SATA (6.93 ul of 5 mg/ml in dimethyl formamide) was added to 2 mg of 7G9IgG protein (12.5 nmol, 0.339 ml of 5.9 mg/ml in PBS) to prepare a reaction mixture having a molar ratio of 1:12 (7G9:SATA). 38.4 ul of HEPES buffer (1M, pH7.4) was added to make the final buffer concentration of 0.1M. The reaction was carried out at 25° C. for 1 hr under Argon with stirring. 7.7 ul of hydroxylamine (1M in Trisbase 1M, 2 mM EDTA) was added to the reaction vessel and the reduction reaction was allowed to proceed for 1 hr without stirring. This generated 7G9-SH in the reaction solution. The reaction mixture was then desalted by passing through PD10 column (10 ml, Amersham) in the conjugation buffer (0.1M citrate pH6, 0.5 mM EDTA, 0.01% Tween80). The PD10 column was previously treated by a solution of Bovine Serum Albumin 1 mg/ml, 10 mM iodoacetamide, 0.2M EDTA, 0.1M glycine, 0.1% Tween in PBS. The column was then equilibrated with 10 column volumes of the conjugation buffer before use. The modified antibody 7G9-SH was eluted in the void volume with the conjugation buffer. The yield of the protein was 95%. The eluted 7G9-SH was diluted to 0.5 mg/ml with the conjugation buffer and used for conjugation immediately.

The 14B7 scAb (see Example 6.2) was derivatized with sSMCC as follows: Sulfo-SMCC (sulfo-succinimidyl-4-(N-maleimidyl) cyclohexane carboxylate, 21.80 ul of 5 mg/ml in water) was added to 2 mg of scAb protein (50 nmol, 0.6897 ml of 2.9 mg/ml in PBS) to prepare a reaction mixture having a molar ratio of 1:5 (scAb:sSMCC). 39.1 ul of HEPES buffer (1M, pH7.4) was added to make a final buffer concentration of 0.1 M. The reaction was carried out at 25° C. for 1 hr under Argon with stirring. The reaction mixture was then desalted in PD10 in the same fashion as above using the conjugation buffer. The yield of the protein was 76%. The eluted scAb-sSMCC was diluted to 0.5 mg/ml and used for conjugation immediately.

The derivatized 7G9 and scAb were mixed such that the scAb-sSMCC to 7G9-SH protein ratio was 1:1 by weight and 3.75:1 by molar amount. Final total protein concentration was 0.5 mg/ml. The conjugation was carried out for 16 hours and was stopped by 50 ug/ml of iodoacetamide. The sample was dialyzed against PBS 5 times at 45 min each in a dialysis bag with molecular weight cutoff of 60 KD. Evaluation of the sample in SDS-PAGE and size exclusion chromatography revealed that the sample was essentially free of free scAb (which has a molecular weight of 40 KD), while almost all 7G9 was in the conjugated form. The total bispecific antibody obtained was 2.81 mg. This represented 70% of conversion of original antibody into the bispecific form. Based on the average molecular weight of the bispecific product scAb-7G9IgG antibody (scAbHP), which was estimated to be 240 KD, there are on average 2 scAb moleclues for each 7G9 molecule in the conjugate scAbHP.

The scAbHP was characterized by several assays. The endotoxin level of the material was determined to be 3.47 mg/mg. The activity of scAbHP binding to red blood cell binding site CR1 (CAA assay, see above) was assayed to be 0.46, indicating the preservation of the binding activity of the original 7G9IgG. The activity of scAbHP binding to Anthrax protective antigen (PA) (PAA assay, see above) was assayed to be 0.28, indicating the preservation of the binding activity of the original scAb. The integrity of the conjugate antibody scAbHP was assayed by the HPCA assay to be 14.69 times that of the standard (14B7-7G9 heteropolymer). This also indicated that the high affinity of scAb antibody fragment was preserved.

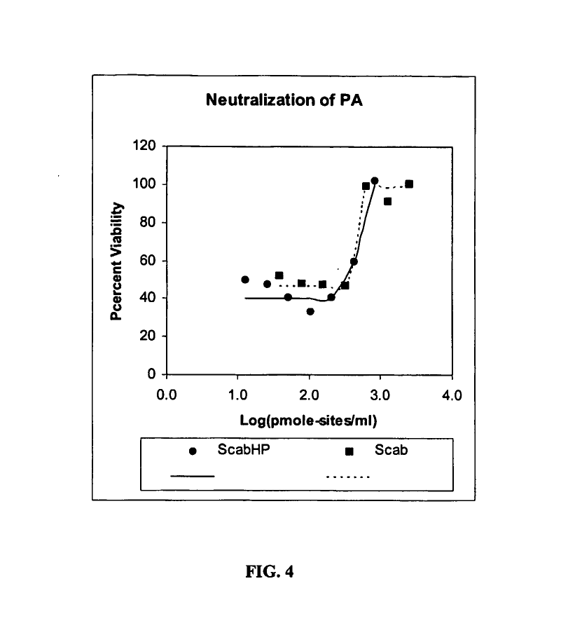

The scAbHP was tested for its PA binding activity in a cell based assay. In this assay, the toxin was used at a level that would kill 48% of the macrophage cell line RAW264.7. The toxin was mixed with various concentrations of the scAbHP or scAb sample for 1 hour. Each mixture was then applied to the cell line RAW264.7. After 4 hours of incubation the percentage of cell surviving the toxin killing was quantified by adding the indicator dye MTT (methylthiazoletetrazolium, Sigma M-5655) and incubated for 1 hour. The cells were lysed and the absorbance at 570 nm was measured. The survival curve is plotted against the concentration of antibody samples used in FIG. 4. The dosage that could protect against 50% of the killing was calculated (ED50). As shown in FIG. 4, the ED50 level of scAbHP was 0.45 nmol binding site/ml. Note that scAbHP has two PA binding sites. The ED50 of scAb, which has one PA binding site, was 0.5 nmol binding site/ml. Therefore, the bispecific scAbHP preserved the binding activity of the scAb molecule.

This scAbHP was tested in a rat toxin challenge model. At time zero, the scAbHP, control scAb, or buffer only was injected iv into the animals. Twelve hours later the animals was challenged by PA (40 ug/rat) and lethal toxin (8 ug/rat). The fate of the animals was observed. All rats in the control group (buffer or scAb only) died within 1-3 hours of toxin challenge. In contrast, the scAbHP treated animal had prolonged survival. Out of 5 rats treated, 2 survived during the observation period of more than 5 days, 2 survived for more than 8 hours, while 1 died at 2 hours. These results indicate that the bispecific scAbHP can protect the animals from Anthrax toxin challenge even 12 hours after the treatment, whereas the nonconjugated scAb, which rapidly clear from the bloodstream of an animal, failed to provide any protection.

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A bispecific molecule comprising an antibody cross-linked to one or more antigen-binding antibody fragments via a chemical cross-linker, wherein said antibody binds a C3b-like receptor, and wherein each of said one or more antigen-binding antibody fragments binds an antigenic molecule.

2. The bispecific molecule of claim 1, wherein said one or more antigen-binding antibody fragments do not comprise an Fc domain.

3. The bispecific molecule of claim 1, wherein said one or more antigen-binding antibody fragments comprise an antigen-binding antibody fragment selected from the group consisting of an Fab, an Fab′, an (Fab′)₂, and an Fv fragment of an immunoglobulin molecule.

4. The bispecific molecule of claim 1, wherein said one or more antigen-binding antibody fragments comprise a single-chain Fv fragment or a single-chain Fv fragment fused with a constant domain of an immunoglobulin molecule.

5. The bispecific molecule of claim 3, wherein at least one of said antigen-binding antibody fragments is a fusion protein further comprising a linker peptide fused to said Fab, Fab′, (Fab′)₂, or Fv fragment, wherein said linker peptide is covalently bound to said chemical cross-linker.

6. The bispecific molecule of claim 1, wherein at least one of said one or more antigen-binding antibody fragments is cross-linked at a predetermined site to said antibody that binds said C3b-like receptor.

7. The bispecific molecule of claim 6, wherein said predetermined site is a cysteine residue in said antigen-binding antibody fragment.

8. The bispecific molecule of claim 7, wherein said predetermined site is the C-terminus of said at least one antigen-binding antibody fragment.

9. The bispecific molecule of claim 1, wherein said antibody that binds a C3b-like receptor is a monoclonal antibody.

10. The bispecific molecule of claim 9, wherein said monoclonal antibody is a murine monoclonal antibody.

11. The bispecific molecule of claim 10, wherein said murine monoclonal antibody is 7G9.

12. The bispecific molecule of claim 9, wherein said monoclonal antibody is a humanized monoclonal antibody.

13. The bispecific molecule of claim 9, wherein said monoclonal antibody is a human monoclonal antibody.

14. The bispecific molecule of claim 9, wherein said one or more antigen-binding antibody fragments bind the protective antigen (PA) protein of Bacillus anthracis (Anthrax).

15. The bispecific molecule of claim 14, wherein said one or more antigen-binding antibody fragments are Fab fragments of murine monoclonal antibody 14B7.

16. The bispecific molecule of claim 14, wherein said one or more antigen-binding antibody fragments are single chain antibody fragments derived from murine monoclonal antibody 14B7.

17. The bispecific molecule of claim 1, wherein said bispecific molecule binds said antigenic molecule with an activity at least 5% of that the antibody from which said antigen-binding antibody fragment is derived.

18. The bispecific molecule of claim 17, wherein said bispecific molecule binds said antigenic molecule with an activity at least 15% of that the antibody from which said antigen-binding antibody fragment is derived.

19. The bispecific molecule of claim 18, wherein said bispecific molecule binds said antigenic molecule with an activity at least 25% of that of the antibody from which said antigen-binding antibody fragment is derived.

20. The bispecific molecule of claim 19, wherein said bispecific molecule binds said antigenic molecule with an activity at least 50% of that of the antibody from which said antigen-binding antibody fragment is derived.

21. The bispecific molecule of claim 20, wherein said bispecific molecule binds said antigenic molecule with an activity at least 90% of that of the antibody from which said antigen-binding antibody fragment is derived.

22. The bispecific molecule of claim 21, wherein said bispecific molecule binds said antigenic molecule with an activity at least 99% of that of the antibody from which said antigen-binding antibody fragment is derived.

23. The bispecific molecule of claim 1, wherein said bispecific molecule binds said antigenic molecule with an activity at least 5% of that of said antigen-binding antibody fragment not cross-linked with said antibody that binds said C3b-like receptor.

24. The bispecific molecule of claim 23, wherein said bispecific molecule binds said antigenic molecule with an activity at least 15% of that of said antigen-binding antibody fragment not cross-linked with said antibody that binds said C3b-like receptor.

25. The bispecific molecule of claim 24, wherein said bispecific molecule binds said antigenic molecule with an activity at least 25% of that of said antigen-binding antibody fragment not cross-linked with said antibody that binds said C3b-like receptor.

26. The bispecific molecule of claim 25, wherein said bispecific molecule binds said antigenic molecule with an activity at least 50% of that of said antigen-binding antibody fragment not cross-linked with said antibody that binds said C3b-like receptor.

27. The bispecific molecule of claim 26, wherein said bispecific molecule binds said antigenic molecule with an activity at least 90% of that of said antigen-binding antibody fragment not cross-linked with said antibody that binds said C3b-like receptor.

28. The bispecific molecule of claim 26, wherein said bispecific molecule binds said antigenic molecule with an activity at least 99% of that of said antigen-binding antibody fragment not cross-linked with said antibody that binds said C3b-like receptor.

29. A method of producing a bispecific molecule, comprising
(a) producing an antigen-binding antibody fragment comprising a cysteine residue by a host cell such that said cysteine residue in said antigen-binding antibody fragment is maintained as a free thiol;
(b) recovering said antigen-binding fragment having said free thiol; and
(c) contacting said antigen-binding antibody fragment having said free thiol with a derivatized antibody that binds a C3b-like receptor under appropriate conditions such that said derivatized antibody cross-links to said antigen-binding antibody fragment at said free thiol;
thereby producing said bispecific molecule.

30. The method of claim 29, wherein said antigen-binding antibody fragment is secreted by said host cell.

31. The method of claim 29, wherein said derivatized antibody that binds a C3b-like receptor is derivatized with a maleimide.

32. The method of claim 31, wherein said maleimide is sulfosuccinimidyl 4-(N-maleimidomethyl) cyclohexane-1-carboxylate.

33. The method of claim 31, wherein said maleimide is NHS-poly(ethylene glycol)-maleimide.

34. A method of producing a bispecific molecule, comprising cross-linking an antibody with an antigen-binding antibody fragment, wherein said antibody binds a C3b-like receptor and said antigen-binding antibody fragment binds an antigenic molecule.

35. A method of producing a bispecific molecule, comprising
(a) producing a thiol-derivatized antigen-binding antibody fragment such that said antigen-binding antibody fragment comprises a free thiol;
(b) producing a maleimide-derivatized antibody that binds a C3b-like receptor such that said antibody comprises a maleimide; and
(c) contacting said antigen-binding antibody fragment containing said free thiol with said antibody containing said maleimide under conditions such that said antibody and said antigen-binding antibody fragment cross-link via said maleimide and said free thiol;
thereby producing said bispecific molecule.

36. The method of claim 35, wherein said antigen-binding antibody fragment is derivatized with N-succinimidyl-S-acetyl-thioacetate (SATA).

37. The method of claim 36, wherein said antigen-binding antibody fragment is derivatized at a molar ratio of about 1:3 to about 1:6 antigen-binding antibody fragment:SATA.

38. The method of claim 35, wherein said antibody that binds a C3b-like receptor is derivatized with sulfosuccinimidyl 4-(N-maleimidomethyl) cyclohexane-1-carboxylate.

39. The method of claim 35, wherein said antibody that binds a C3b-like receptor is derivatized with NHS-poly(ethylene glycol)-maleimide.

40. The method of claim 35, wherein said step (c) is carried out by a method comprising mixing said thiol-derivatized antigen-binding antibody fragment and said maleimide-derivatized antibody that binds a C3b-like receptor at a molar ratio of about 1:1.

41. The method of claim 35, wherein said step (c) is carried out by a method comprising mixing said thiol-derivatized antigen-binding antibody fragment and said maleimide-derivatized antibody that binds a C3b-like receptor at a molar ratio of about 2:1.

42. The product as produced by the method of claim 34.

43. A polyclonal population of bispecific molecules comprising a plurality of different bispecific molecules, each bispecific molecule in said plurality comprising an antibody cross-linked via a chemical cross-linker to one or more antigen-binding antibody fragments, wherein said antibody binds a C3b-like receptor, and wherein said antigen-binding fragments bind an antigenic molecule.

44. The polyclonal population of bispecific molecules of claim 43, wherein said one or more antigen-binding antibody fragments do not comprise an Fc domain.

45. The polyclonal population of bispecific molecules of claim 43, wherein said one or more antigen-binding antibody fragments comprise an antigen-binding antibody fragment selected from the group consisting of an Fab, an Fab′, an (Fab′)₂, and an Fv fragment of an immunoglobulin molecule.

46. The polyclonal population of bispecific molecules of claim 43, wherein said one or more antigen-binding antibody fragments comprise a single-chain Fv fragment or a single-chain Fv fragment fused with a constant domain of an immunoglobulin molecule.

47. The polyclonal population of bispecific molecules of claim 45, wherein at least one of said antigen-binding antibody fragments is a fusion protein further comprising a linker peptide fused to said Fab, Fab′, (Fab′)₂, or Fv fragment, wherein said linker peptide is covalently bound to said chemical cross-linker.

48. The bispecific molecule of any one of claim 1, wherein said antigenic molecule is a molecule desired to be removed from the circulation of a mammal.

49. The bispecific molecule of claim 48, wherein said mammal is a human, and wherein said antibody binds CR1.

50. The bispecific molecule of claim 1, wherein said antigenic molecule is an antigen of a pathogen, and wherein said antibody binds CR1.

51. The bispecific molecule of claim 50, wherein said pathogen is a bacterium.

52. The bispecific molecule of claim 50, wherein said pathogen is a virus.

53. The bispecific molecule of claim 1, wherein said antigenic molecule is a toxin.

54. A method of treating a mammal having an undesirable condition associated with the presence of an antigenic molecule in its circulation, comprising the step of administering to the mammal a therapeutically effective amount of a bispecific molecule comprising an antibody cross-linked to one or more antigen-binding antibody fragments via a chemical cross-linker, wherein said antibody binds a C3b-like receptor on a blood cell of said mammal, and wherein each of said one or more antigen-binding antibody fragments binds said antigenic molecule.

55. The method of claim 54, wherein said one or more antigen-binding antibody fragments do not comprise an Fc domain.

56. The method of claim 54, wherein said one or more antigen-binding antibody fragments comprise an antigen-binding antibody fragment selected from the group consisting of an Fab, an Fab′, an (Fab′)₂, and an Fv fragment of an immunoglobulin molecule.

57. The method of claim 54, wherein said one or more antigen-binding antibody fragments comprise a single-chain Fv fragment or a single-chain Fv fragment fused with a constant domain of an immunoglobulin molecule.

58. The method of claim 54, wherein at least one of said antigen-binding antibody fragments is a fusion protein further comprising a linker peptide fused to said Fab, Fab′, (Fab′)₂, or Fv fragment, wherein said linker peptide is covalently bound to said chemical cross-linker.

59. The method of claim 54, wherein said antibody that binds a C3b-like receptor is a monoclonal antibody.

60. The method of claim 59, wherein said monoclonal antibody is a murine monoclonal antibody.

61. The method of claim 60, wherein said murine monoclonal antibody is 7G9.

62. The method of claim 59, wherein said monoclonal antibody is a humanized monoclonal antibody.

63. The method of claim 59, wherein said monoclonal antibody is a human monoclonal antibody.

64. The method of claim 60, wherein said one or more antigen-binding antibody fragments bind the protective antigen (PA) protein of Bacillus anthracis (Anthrax).

65. The method of claim 60, wherein said one or more antigen-binding antibody fragments are Fab fragments of murine monoclonal antibody 14B7.

66. The method of claim 60, wherein said one or more antigen-binding antibody fragments are single chain antibody fragments derived from murine monoclonal antibody 14B7.

67. The method of claim 54, wherein said mammal is a human, and wherein said antibody binds CR1.

68. The method of claim 67, wherein said antigenic molecule is an antigen of a pathogen.

69. The method of claim 68, wherein said pathogen is a bacterium.

70. The method of claim 68, wherein said pathogen is a virus.

71. The method of claim 67, wherein said antigenic molecule is a toxin.

72. The method of claim 54, wherein said bispecific molecule binds said antigenic molecule with an activity at least 5% of that the antibody from which said antigen-binding antibody fragment is derived.

73. The method of claim 72, wherein said bispecific molecule binds said antigenic molecule with an activity at least 15% of that the antibody from which said antigen-binding antibody fragment is derived.

74. The method of claim 73, wherein said bispecific molecule binds said antigenic molecule with an activity at least 25% of that of the antibody from which said antigen-binding antibody fragment is derived.

75. The method of claim 74, wherein said bispecific molecule binds said antigenic molecule with an activity at least 50% of that of the antibody from which said antigen-binding antibody fragment is derived.

76. The method of claim 75, wherein said bispecific molecule binds said antigenic molecule with an activity at least 90% of that of the antibody from which said antigen-binding antibody fragment is derived.

77. The method of claim 76, wherein said bispecific molecule binds said antigenic molecule with an activity at least 99% of that of the antibody from which said antigen-binding antibody fragment is derived.

78. The method of claim 54, wherein said bispecific molecule binds said antigenic molecule with an activity at least 5% of that of said antigen-binding antibody fragment not cross-linked with said antibody that binds said C3b-like receptor.

79. The method of claim 78, wherein said bispecific molecule binds said antigenic molecule with an activity at least 15% of that of said antigen-binding antibody fragment not cross-linked with said antibody that binds said C3b-like receptor.

80. The method of claim 79, wherein said bispecific molecule binds said antigenic molecule with an activity at least 25% of that of said antigen-binding antibody fragment not cross-linked with said antibody that binds said C3b-like receptor.

81. The method of claim 80, wherein said bispecific molecule binds said antigenic molecule with an activity at least 50% of that of said antigen-binding antibody fragment not cross-linked with said antibody that binds said C3b-like receptor.

82. The method of claim 81, wherein said bispecific molecule binds said antigenic molecule with an activity at least 90% of that of said antigen-binding antibody fragment not cross-linked with said antibody that binds said C3b-like receptor.

83. The method of claim 82, wherein said bispecific molecule binds said antigenic molecule with an activity at least 99% of that of said antigen-binding antibody fragment not cross-linked with said antibody that binds said C3b-like receptor.

84. A pharmaceutical composition for treating a mammal having an undesirable condition associated with the presence of an antigenic molecule in its circulation, comprising a therapeutically effective amount of the bispecific molecule of any one of claims 1-5 and 9-16 and a pharmaceutically acceptable carrier.

85. A method of producing a bispecific molecule, comprising
(a) producing a maleimide-derivatized antigen-binding antibody fragment such that said antigen-binding antibody fragment comprises a maleimide;
(b) producing a thiol-derivatized antibody that binds a C3b-like receptor such that said antibody comprises a free thiol; and
(c) contacting said antigen-binding antibody fragment containing said maleimide with said antibody containing said free thiol under conditions such that said antibody and said antigen-binding antibody fragment cross-link via said maleimide and said free thiol,
thereby producing said bispecific molecule.

86. The method of claim 85, wherein said antigen-binding antibody fragment is derivatized with sulfosuccinimidyl 4-(N-maleimidomethyl) cyclohexane-1-carboxylate (sSMCC).

87. The method of claim 86, wherein said antigen-binding antibody fragment is derivatized at a molar ratio of about 1:5 antigen-binding antibody fragment:sSMCC.

88. The method of claim 85, wherein said antibody that binds a C3b-like receptor is derivatized with N-succinimidyl-S-acetyl-thioacetate (SATA).

89. The method of claim 87, wherein said antibody that binds a C3b-like receptor is derivatized with N-succinimidyl-S-acetyl-thioacetate (SATA) at a molar ratio of about 1:12 antibody:SATA.

90. The method of claim 89, wherein said step (c) is carried out by a method comprising mixing said maleimide-derivatized antigen-binding antibody fragment and said thiol-derivatized antibody that binds a C3b-like receptor at a molar ratio of about 3.75:1 maleimide-derivatized antigen-binding antibody fragment:thiol-derivatized antibody.

91. The bispecific molecule as produced by the method of claim 85.

92. The bispecific molecule of claim 91, wherein said antigen-binding antibody fragment is 14B7scAb and said antibody that binds a C3b-like receptor is the murine monoclonal antibody 7G9, and wherein said bispecific molecule has two 1 4B7scAb cross-linked to a 7G9.

93. A method of treating or preventing Anthrax infection in an animal, comprising administering to said animal a therapeutically or prophylactically sufficient amount of the bispecific molecule of claim 91.
